(19) United States
(12) Patent Application Publication
(10) Pub. No.: US 2003/0105343 A1
(43) Pub. Date: Jun. 5, 2003

(54) Benzofuran compounds

(76) Inventors: Peter Neil Taylor, Oxford (GB); Sally Anderson, Oxford (GB); Christopher James Booth, Berkshire (GB); Geraldine Laura Ballantyne Verschoor, Oxford (GB)

Correspondence Address: Neil A. Duchez, Renner, Otto, Boisselle & Sklar, LLP, Nineteenth Floor, 1621 Euclid Avenue, Cleveland, OH, 44115, US

(21) Appl. No.: 10/254,439

(22) Filed: Sep. 25, 2002

(30) Foreign Application Priority Data
Sep. 28, 2001 (GB) 0123413.7

(51) Int. Cl. C 07D 4 7/14

(52) U.S. Cl. 549/462

(57) ABSTRACT

There is disclosed a method of preparing a branched benzofuran compound comprising a core moiety which contains at least one aromatic ring and which has at least three substituted or unsubstituted benzofuran groups covalently linked thereto. The method comprises the steps of

(i) forming an intermediate ethynylene compound in which at least three benzene rings are each linked to the core moiety via an ethynylene bond, and where each benzene ring is substituted at the ortho position (relative to the position of the ethynylene bond) by a blocked carbonyloxy group,

(ii) deblocking the carbonyloxy groups, and

(iii) effecting ring closure by reaction between the deblocked carbonyloxy groups and the adjacent ethynylene bonds to form the furan rings of the benzofuran groups, whereby to produce the branched benzofuran compound.

### BACKGROUND OF THE INVENTION

1. Field of the Invention

This invention relates to benzofuran compounds and is particularly concerned with branched benzofuran compounds which are suitable for charge transport and emission inorganic electroluminescent devices and organic lasers. The compounds also have potential use in photovoltaic devices and organic thin film transistors.

2. Description of the Related Art

Organic electroluminescent devices are based on the principle that current injected into an emitter material results in the formation of an energetically excited state. The excited state may then decay to its ground state with the emission of light.

Research into the use of organic materials for electroluminescence started in the 1960s. Early attempts used single crystals of organic materials, voltages in excess of 100 V were required to inject sufficient charge to achieve significant light output [Helfrich, W. et al, Phys. Rev. Lett, 1965, 14, 229]. A major breakthrough came in 1987 when layers of two different organic materials were incorporated into a device. One material acts as a hole transport agent while the other is used for electron transport [Tang, C. W. et al, Appl. Phys. Lett.1987, 51, 913]. Further improvements in colour purity and device efficiency were made by doping the electron transport layer with an emissive dye [Tang, C. W. et al, J. Appl. Phys. 1989, 65, 3610].

Benzofurans have been used for organic electroluminescence. For example benzofuran moieties have been appended to spirobifluorenes [U.S. Pat. No. 5,840,217], other aromatic cores [U.S. Pat. No. 5,077,142, JP 6145658, JP 6107648, JP 6092947, JP 6065567, JP 6228558, EP-A-0999256 and JP 2000192028] and incorporated into polymethine dyes which when fluorescent, may be used in organic electroluminescent devices [U.S. Pat. Nos. 4,948,893 and 4,900,831].

The preparation of a branched benzofuran is disclosed by Buu-Hoi, N. P. et al, Acad. Sc. Paris, 1966, 263, 1237-1239.

A process for the preparation of monomeric benzofurans by performing a ring closing operation to form the furan moiety is disclosed in Tetrahedron, 1995, 51(30), 8199-8212.

### SUMMARY OF THE INVENTION

According to one aspect of the present invention, there is provided a method of preparing a branched benzofuran compound comprising a core moiety which contains at least one aromatic ring and which has at least three substituted or unsubstituted benzofuran groups covalently linked thereto, said method comprising the steps of

(i) forming an intermediate ethynylene compound in which at least three benzene rings are each linked to the core moiety via an ethynylene bond, and where each benzene ring is substituted at the ortho position (relative to the position of the ethynylene bond) by a blocked carbonyloxy group,

(ii) deblocking the carbonyloxy groups, and

(iii) effecting ring closure by reaction between the deblocked carbonyloxy groups and the adjacent ethynylene bonds to form the furan rings of the benzofuran groups, whereby to produce the branched benzofuran compound.

The intermediate ethynylene compound formed in step (i) may be prepared by forming ethynylene groups on the core moiety and then reacting each of these with a reactive substituent on a benzene ring which is substituted at the ortho position (relative to the position of the reactive substituent) by a blocked carbonyloxy group.

An example of a suitable preparation route involving this procedure is set out below:—

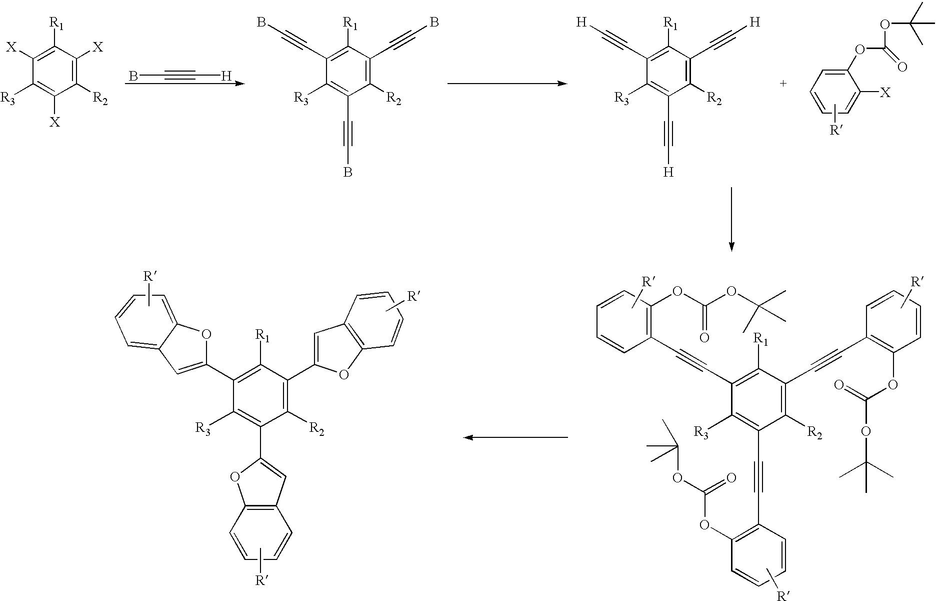

wherein

R₁, R₂, R₃ and R′ are independently selected from H, an aliphatic group (eg a substituted or unsubstituted alkyl or alkoxy group), an aryl group (eg substituted or unsubstituted phenyl), a halogen such as F, CN and NO₂, B is a blocking group such as a trimethylsilyl group), and X is a reactive group, e.g. a halogen group such as Br or I.

Alternatively, the intermediate ethynylene compound formed in step (i) may be prepared by preparing compounds in which a benzene ring is substituted with an ethynylene group and a blocked carbonyloxy group which are in the ortho position with respect to each other, and then reacting the ethynylene groups of said compounds with reactive groups on the core moiety so as to link each of the benzene rings with the core moiety via an ethynylene bond.

An example of a suitable preparation route involving this alternative method is set out below:—

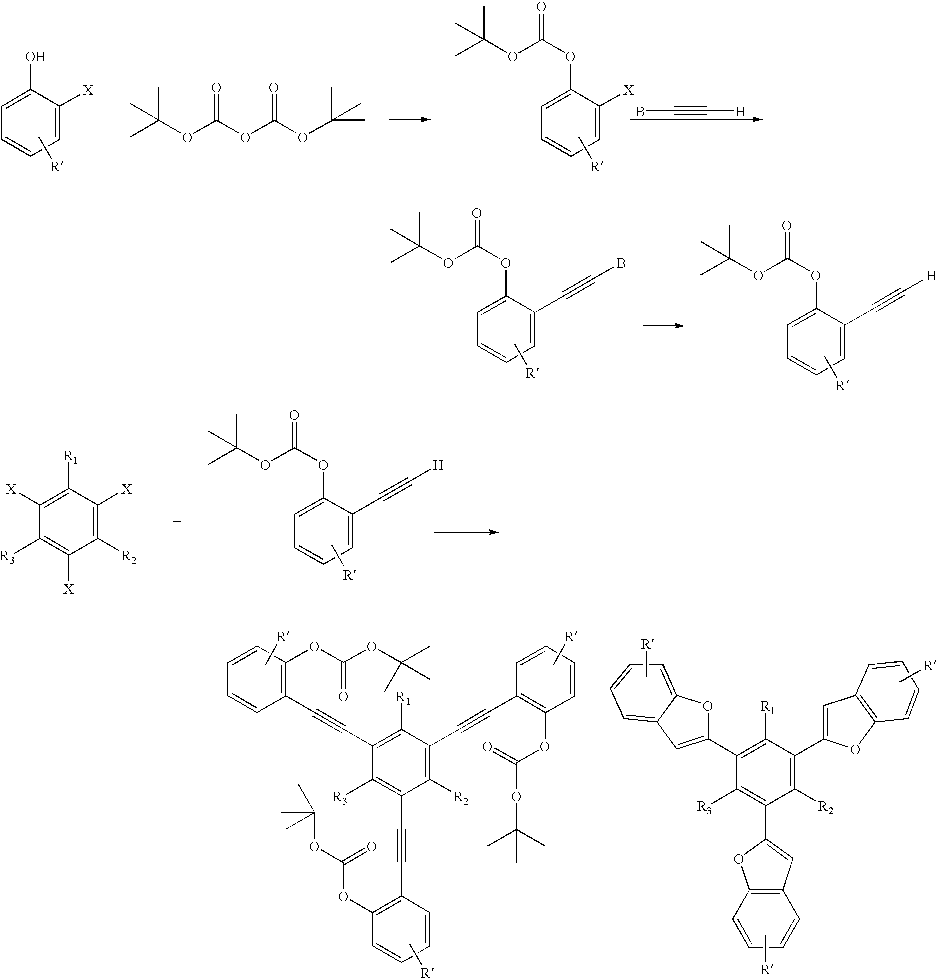

wherein

R₁, R₂, R₃ and R′ are independently selected from H, an aliphatic group (eg a substituted or unsubstituted alkyl or alkoxy group), an aryl group (eg substituted or unsubstituted phenyl), a halogen such as F, CN and NO₂, B is a blocking group such as a triisopropylsilyl group, and X is a reactive group, e.g. a halogen group such as I.

In the above examples, the core moiety is

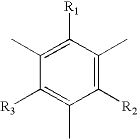

In alternative embodiments, the core moiety is

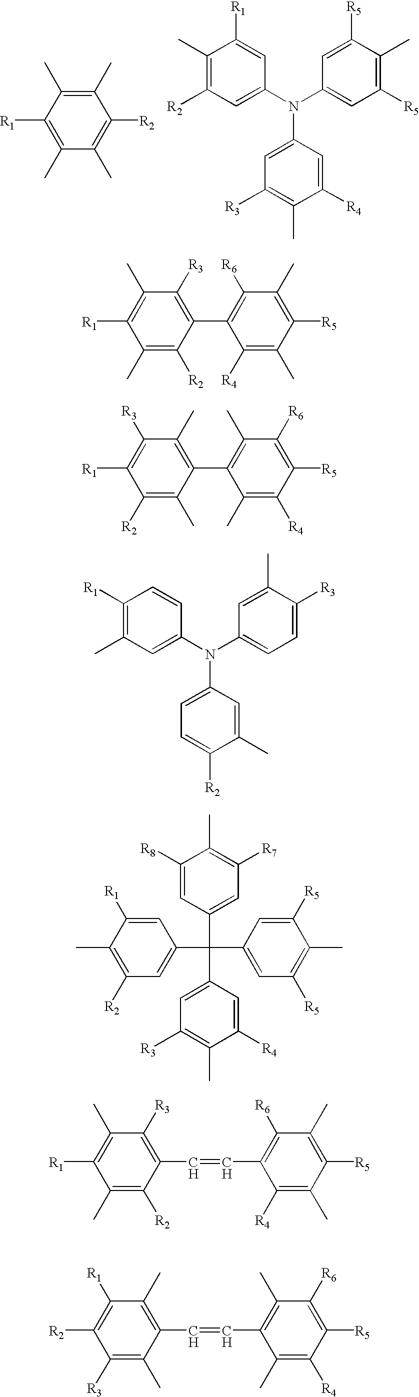

wherein

R₁ to R₁₂ are independently selected from H, an aliphatic group (eg a substituted or unsubstituted alkyl or alkoxy group), an aryl group (eg substituted or unsubstituted phenyl), a halogen such as F, CN and NO₂, and A is O, S, or NR (where R is selected from the moieties defined above for R₁ to R₁₂). Also included are core moieties of any of the above ring structures where the bonds for linking to the benzofuranyl groups are at any other positions on the respective rings, provided that there are at least three such bonds in all, with the R₁ to Rₓ substituents being correspondingly positioned on the respective rings.

Compounds containing such core moieties can be prepared using analogous procedures to those described above. For example, compounds containing a core moiety cased on tetraphenylmethane can be prepared using the following reaction scheme:—

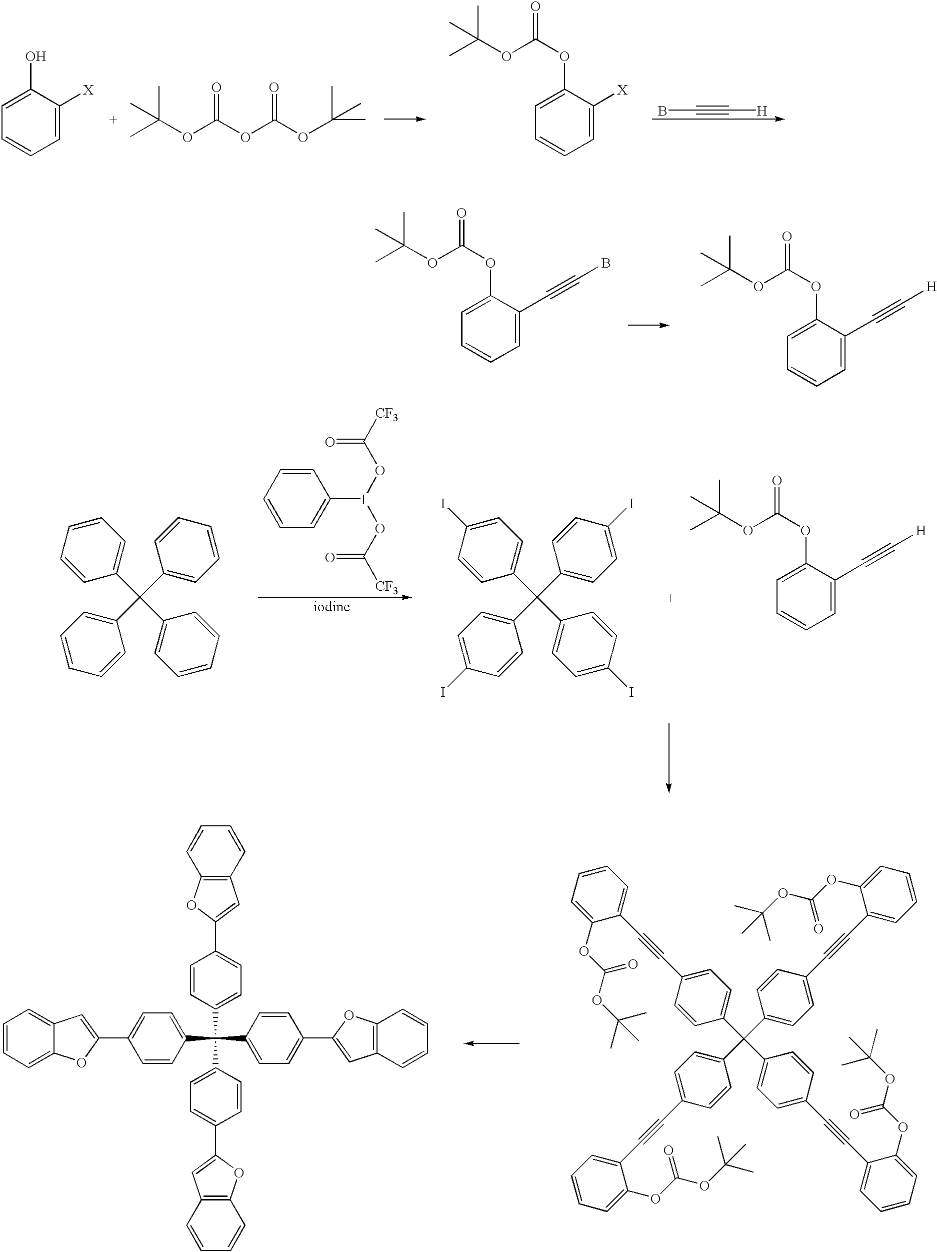

It will be understood that the number of benzofuran groups which may be linked to the above identifed core moieties maybe different to that indicated by the dangling bonds.

Some examples of the types of compound which can be prepared by the method of the present invention are as follows:—

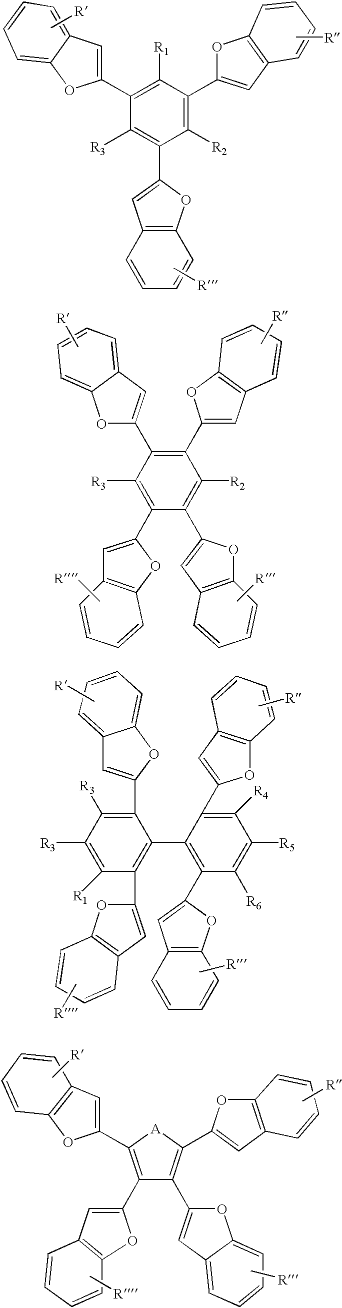

where

each of R₁ to R₈ is independently selected from H, an aliphatic group, an aromatic group, a halogen, CN and NO₂, and each of R′ to R″″ is independently selected from at least one of H, an aliphatic group, an aromatic group, a halogen, CN and NO₂, and A is O, S or NR (where R is selected from the moieties defined above for R₁ to R₈)

According to another aspect of the present invention, there is provided a compound having one of the following general formulae:—

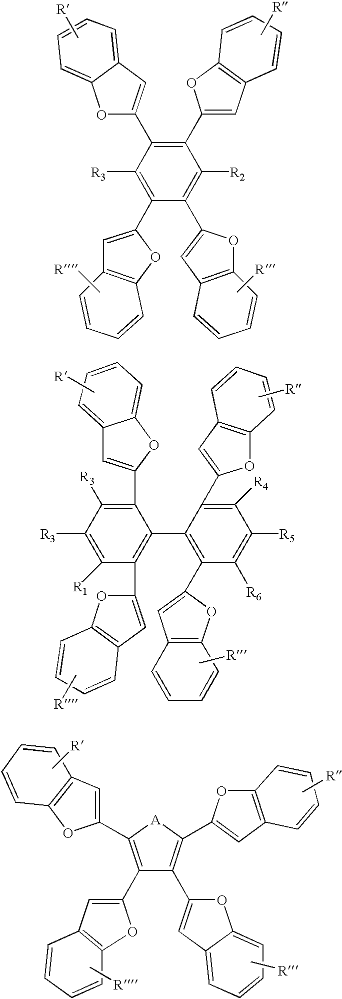

where

each of R₁ to R₈ is independently selected from H, an aliphatic group, an aromatic group, a halogen, CN and NO₂, and each of R′ to R″″ is independently selected from at least one of H, an aliphatic group, an aromatic group, a halogen, CN and NO₂, and A is O, S or NR (where R is selected from the moieties defined above for R₁ to R₈)

Such compounds can be produced by the method according to said one aspect of the present invention or they can be produced by another method.

### DESCRIPTION OF THE PREFERRED EMBODIMENTS

The present invention will now be described in further detail in the following Examples.

### EXAMPLE 1

### Preparation of tetra(p-benzofuranylphenyl)methane

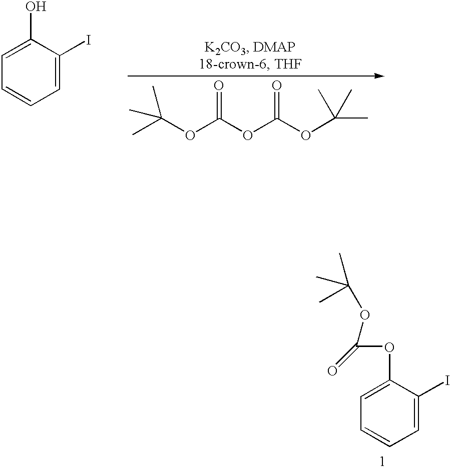

Di-tert-butyl dicarbonate (33 g, 1.5×10⁻¹ moles) was added to a mixture of 2-iodophenol (30 g, 1.36×10⁻¹ moles), potassium carbonate (27 g, 1.95×10⁻¹ moles), dimethylaminopyridine (catalytic amount) and 18-crown-6 (catalytic amount) in dry tetrahydrofuran (200 ml). After stirring at room temperature for 1 hour, the reaction was quenched by the addition of brine and the resulting mixture extracted with diethyl ether. The organic fractions were then dried over magnesium-sulphate and evaporated. The pale yellow oil was purified by flash chromatography (SiO₂ hexanes:dichloromethane (3:1)) and then distillation (bp. 80° C., 0.5 mbar) to give compound 1, a colourless oil (40 g, 90%).

¹H NMR (300 MHz, CDCl₃) 7.82 (dd, J=1, 8 Hz 1 H), 7.37 (ddd, J=1, 8, 8 Hz, 1 H), 7.17 (dd, J=1, 8 Hz, 1H), 6.99 (ddd, J=1, 8, 8 Hz, 1 H), 1.59 (s, 9 H).

¹³C NMR (75 MHz, CDCl₃) 151.59, 151.17, 139.69, 129.74, 127.88, 123.06, 90,83, 84.40, 27.94.

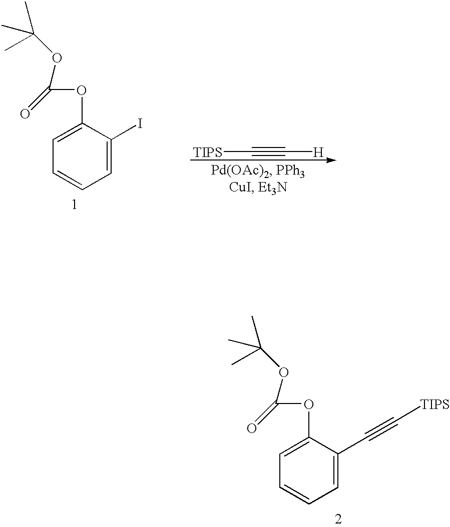

(Tri-iso-propylsilyl)acetylene (7.11 g, 3.9×10⁻² moles) was added to a degassed mixture of the aryliodide compound 1 (10 g, 3.12×10⁻² moles), palladium(II)acetate (136 mg, 6.1×10⁻⁴ moles), copper(I) iodide (60 mg, 3.1×10⁻⁴ moles), triphenylphosphine (326 mg, 1.2×10⁻³ moles) and dry triethylamine (80 ml). The mixture was degassed briefly and then heated at 70° C. overnight. A thick yellow precipitate formed which was filtered off and washed with hexanes. The combined filtrates were evaporated and the resulting oil purified by flash chromatography (SiO₂, hexanes:dichloromethane (4:1)) to give a clear oil (11.4 g, 97%).

¹H NMR (300 MHz, CDCl₃) 7.52 (dd, J=1, 8 Hz, 1 H), 7.33 (ddd, J=1, 8, 8 Hz, 1 H), 7.18 (ddd, J=1, 8, 8 Hz, 1 H) 7.17 (dd, J=1, 8 Hz, 1H), 1.54 (s, 9 H), 1.14 (s, 21 H).

¹³C NMR (75 MHz, CDCl₃) 151.93, 151.70, 134.10, 129.53, 125.94, 122.52, 117.80, 101.43, 96.34, 84.67, 27.86.18.90, 11.47.

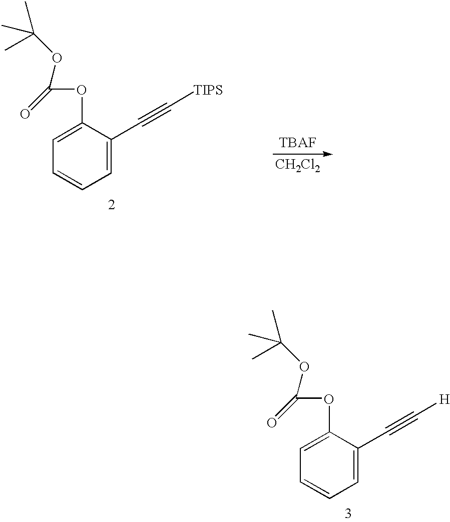

The tri-iso-propylsilyl protected acetylene compound 2 (21.1 g, 5.6×10⁻² moles) was dissolved in dichloromethane (800 ml), and tetrabutylammonium fluoride (1 M in THF, 56 ml, 5.6×10⁻² moles) was added. The reaction was stirred at room temperature for 15 minutes, then quenched by the addition of calcium chloride and brine. The product was extracted with dichloromethane, the organic fractions were dried over magnesium sulphate and then the solvent evaporated. The resulting oil was purified by flash chromatography (SiO₂, hexanes: dichloromethane (3:1)) and then distillation (bp. 65° C., 0.05 mbar) to give a white waxy solid (10.8 g, 88%).

¹H NMR (300 MHz, CDCl₃) 7.54 (dd, J=1, 8 Hz 1 H), 7.38 (ddd, J=1, 8, 8 Hz, 1 H), 7.21 (ddd, J=1, 8, 8 Hz, 1H), 7.18 (dd, J=1, 8 Hz, 1 H), 3.28 (s, 1 H), 1.57 (s, 9 H).

¹³C NMR (75 MHz, CDCl₃) 152.54, 151.41, 133.86, 130.28, 126.12, 122.27, 116.63, 84.13, 82.54, 78.55, 27.88.

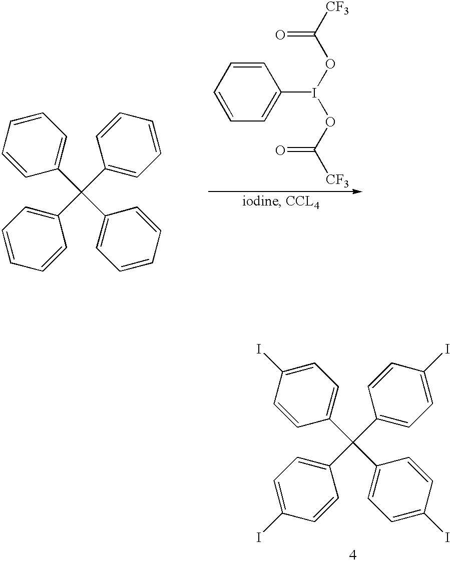

Following the general procedure of Tett. Lett. 1997, 1485, a suspension of tetraphenylmethane (2.0 g, 6.2×10⁻³ moles), [bis(trifluoroacetoxy)iodo]benzene (6.23 g, 1.5×10⁻²moles) and iodine (3.3 g, 1.30×10⁻² moles) in carbon tetrachloride (40 ml) was heated at 60° C. After 1 hour, the iodine colour disappeared and a thick precipitate formed; this was filtered off and washed with ethanol followed by acetone. The solid was recrystallised from tetrahydrofuran to give pale yellow platelets (2.0 g, 39%) of the tetraiodide compound 4.

¹H NMR (300 MHz, CDCl₃) 7.59 (d, J=8, 8 H), 6.89 (d, J=8, 8 H).

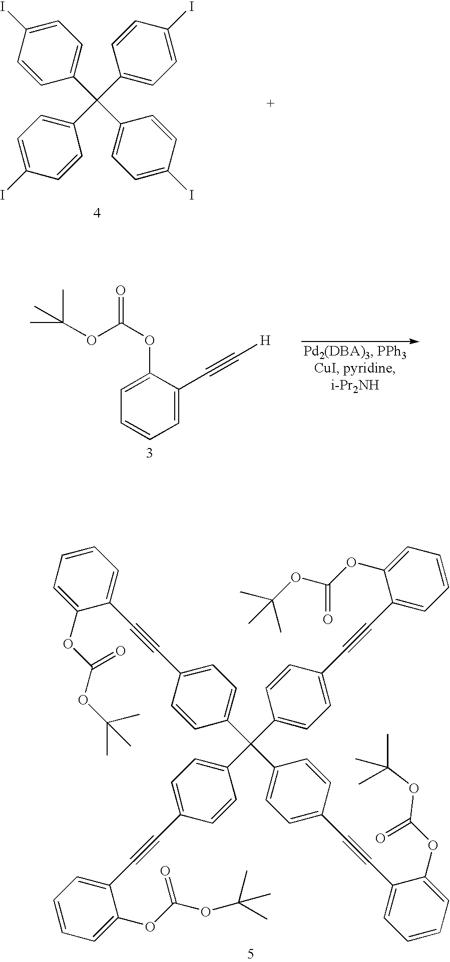

The tetraiodide compound 4 (1.98 g, 2.4×10⁻³ moles), the phenylacetylene compound 3 (2.6 g, 1.2×10⁻² moles), copper(I) iodide (46 mg, 2.4×10⁻⁴ moles) and triphenylphosphine (504 mg, 1.92×10⁻³ moles) were dried under vacuum and flushed-with nitrogen. A mixture of dry pyridine (40 ml) and di-iso-propylamine (10 ml) was degassed and tris(dibenzylideneacetone)dipalladium (220 mg, 2.4×10⁻⁴ moles) was added. After a further degassing, this mixture was transferred via cannular to the flask containing the other reagents and heated at 50° C. overnight.

The mixture was filtered through a short plug of SiO₂ eluting with dichloromethane before purification by flash chromatography (SiO₂, dichloromethane:cyclohexane:triethylamine(3:1:0.01). Recrystallisation from toluene and cyclohexane gave fine needles of compound 5 (2.32 g, 82%).

¹H NMR (300 MHz, CDCl₃) 7.57 (dd, J=1, 8 Hz, 4 H), 7.46 (d, J=8 Hz, 8 H), 7.37 (ddd, J=1, 8, 8 Hz, 4 H), 7.25-7.7.18 (m, 8 H), 7.18 (d, J=8 Hz, 8 H), 1.52 (s, 36 H).

¹³C NMR (75 MHz, CDCl₃) 151.87, 151.56, 146.26, 133.26, 131.35, 132.05, 129.82, 126.20, 122.32, 121.48, 117.69, 94,21, 84.81, 83.94, 65.15, 27.91.

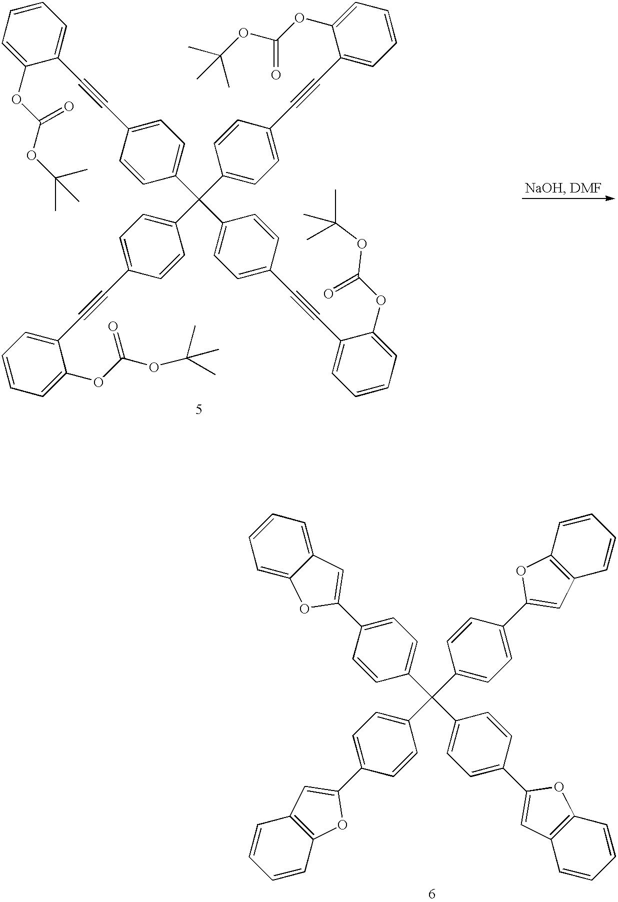

A suspension of the phenylacetylene compound5 (1.0 g, 8.4×10⁻⁴) and sodium hydroxide (200 mg, 5.0×10⁻³ moles) in N,N-dimethylformamide (50 ml) was degassed then heated at reflux overnight. The solvent was distilled under reduced pressure and the residue extracted with methanol. The insoluble material was separated by centrifugation and washed with methanol (3×20 ml). The resulting solid was dried and then recrystallised from toluene to give the desired compound 6 in the form of a bright white powder (566 mg, 85%)

¹H NMR (300 MHz, CD₂Cl₂) 7.84 (d, J=8 Hz 8 H), 7.60 (dd, J=1, 8 Hz, 4 H), 7.52 (dd, J=1, 8 Hz, 4H), 7.47 (d, J=8 Hz, 8 H), 7.29 (ddd, J=1, 8, 8 Hz, 4 H), 7.23 (dd, J=1, 8 Hz, 4 H), 7.07 (s, 4 H).

### EXAMPLE 2

### Preparation of 1,3,5-tribenzofuranylbenzene

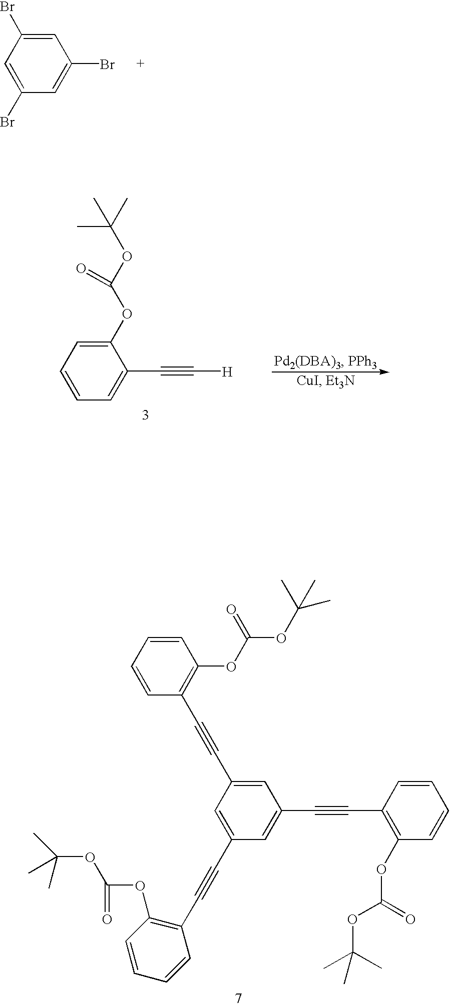

1,3,5-Tribromobenzene (1.25 g, 4.0×10⁻³ moles), the phenylacetylene compound 3 (3.3 g, 1.5×10⁻² moles), copper(I) iodide (57 mg, 3.0×10⁻⁴ moles) and triphenylphosphine (629 mg, 2.4×10⁻² moles) were dried under vacuum and flushed with nitrogen. Dry triethylamine (50 ml) was degassed and tris(dibenzylideneacetone)dipalladium (275 mg, 3.0×10⁻⁴ moles) was added. The mixture was degassed twice, transferred via cannular to the flask containing the other reagents and then heated at 60° C. for 3 hours. TLC analysis suggested the reaction was incomplete so a further portion of the phenylacetylene compound3 (600 mg, 2.75×10⁻³ moles) was added and the mixture was stirred overnight at 70° C. The mixture was filtered through a short plug of SiO₂, washing with dichloromethane before purification by flash chromatography (SiO₂, dichloromethane:cyclohexane:triethylamine (1:1:0.01). Recrystallisation from cyclohexane gave white crystals of compound 7 (2.8 g, 96%).

¹H NMR (300 MHz, CDCl₃) 7.67 (s, 3 H) 7.56 (dd, J=1, 8 Hz, 3 H), 7.41 (ddd, J=1 8, 8 Hz, 3 H), 7.27 (ddd, J=1, 8, 8 Hz, 3 H), 7.22 (dd, J=1, 8 Hz, 3H) 1.55 (s, 27 H).

¹³C NMR (75 MHz, CDCl₃) 152.10, 151.63, 134.49, 133.15, 130.20, 126.25, 124.03, 122.43, 117.31, 92.77, 85.67, 84,23, 27.91.

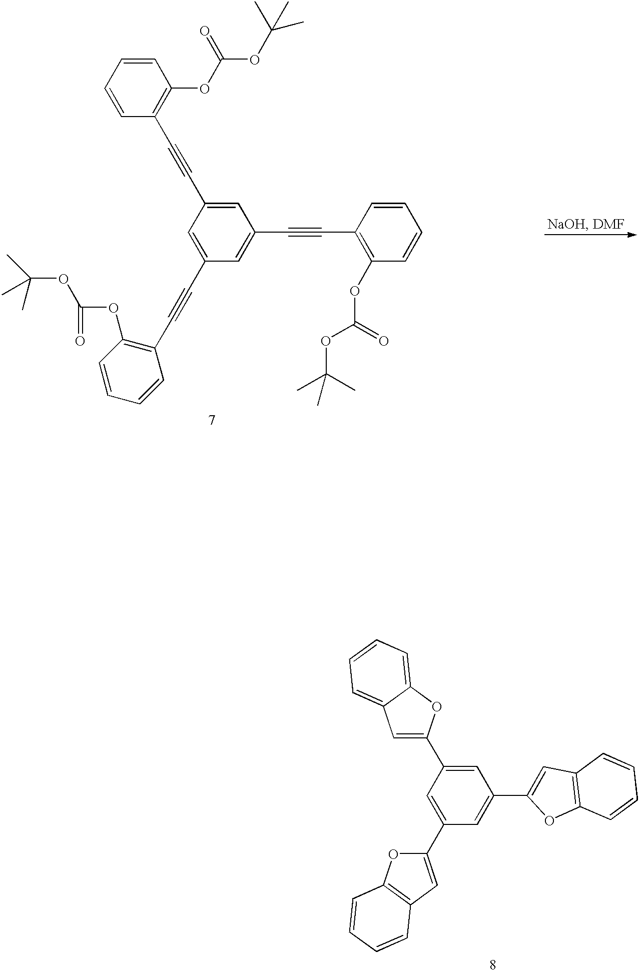

The phenylacetylene compound 7 (1.8 g, 2.5×10⁻³ moles) and sodium hydroxide (400 mg, 1.0×10⁻² moles) in N,N-dimethylformamide (50 ml) was degassed then heated at reflux overnight. The solvent was distilled under reduced pressure and the residue extracted with methanol.

The insoluble material was separated by centrifugation and washed with methanol (3×20 ml). The resulting solid was dried and then purified by sublimation under reduced pressure (230° C., 10⁻⁴ mbar) to give the desired compound 8 in the form of a bright white solid (702 mg, 66%).

¹H NMR (300 MHz, CDCl₃) 8.34 (s, 3 H), 7.67 (dd, J=1, 8 Hz, 3 H), 7.63 (dd, J=1, 8 Hz, 3 H), 7.37 (ddd, J=1, 8, 8 Hz, 3 H), 7.30 (ddd, J=1, 8, 8 Hz, 3 H), 7.29 (s, 3 H).

¹³C NMR (75 MHz, CDCl₃) 155.23, 155.11, 131.89, 129.27, 124.99, 123.39, 121.40, 121.24, 111.55, 102.79.

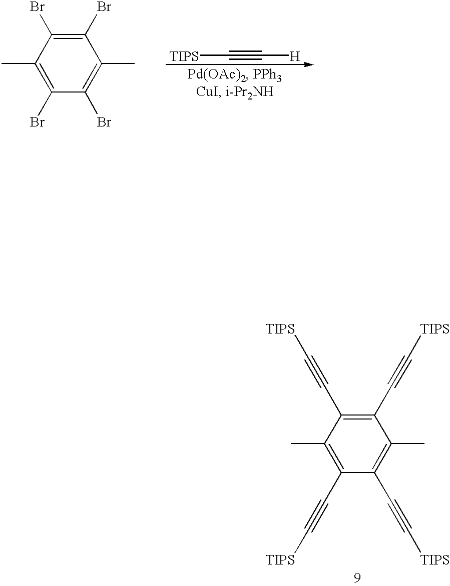

### EXAMPLE 3

### Preparation of 1,2,4,5-tetra(benzofuranyl)-p-xylene

A mixture of 1,2,4,5-tetrabromo-p-xylene (2.8 g, 6.6×10⁻³moles), palladium(II) acetate (449 mg, 2.0×10⁻³), triphenylphosphine (2.1 g, 8.0×10⁻³ moles), copper(I) iodide (190 mg, 1.0×10⁻³moles) and di-iso-propylamine (100 ml) was thoroughly degassed and flushed with nitrogen. (Tri-iso-propylsilyl)acetylene (14.6 g, 8.0×10⁻² moles) was added and after a brief degassing the mixture was heated to 50° C. for two hours and then at 70° C. overnight. TLC analysis of the mixture suggested the reaction was incomplete so tris(dibenzylideneacetone)dipalladium (250 mg, 2.7×10⁻⁴ moles) was added and the mixture was then briefly degassed before heated at reflux overnight. The mixture was cooled and filtered. After removing the solvent under reduced pressure, the resulting oil was purified by flash chromatography (SiO₂, hexanes) Recrystallisation from ethanol gave white crystals of the acetylene compound 9 (1.8 g, 33%).

¹H NMR (300 MHz, CDCl₃) 2.59 (s, 6 H), 1.15 (s, 84 H).

¹³C NMR (75 MHz, CDCl₃) 142.19, 125.29, 104.33, 101.18, 20.45, 19.03, 11.65.

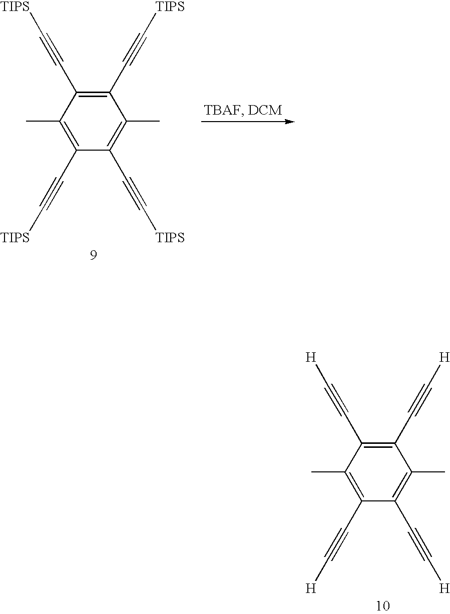

The tri-iso-propylsilyl protected acetylene compound 9 (1.8 g, 2.2×10⁻³ moles) was dissolved in dichloromethane (150 ml) and tetrabutylammonium fluoride (1 M in THF, 8.8 ml, 8.8×10⁻³ moles) was added. The reaction was stirred at room temperature for 15 minutes, then quenched by the addition of calcium chloride and brine. The product was extracted with dichloromethane, the organic fractions were dried over magnesium sulphate and then the solvent evaporated. The resulting oil was purified by flash chromatography (SiO₂, cyclohexane:dichloromethane (3:1) to give compound 10 as a pink solid (390 mg, 88%).

¹H NMR (300 MHz, CDCl₃) 3.63 (s, 4 H), 2.59 (s, 6 H).

¹³C NMR (75 MHz, CDCl₃) 141.62, 125.52, 86.87, 80.76, 19.81.

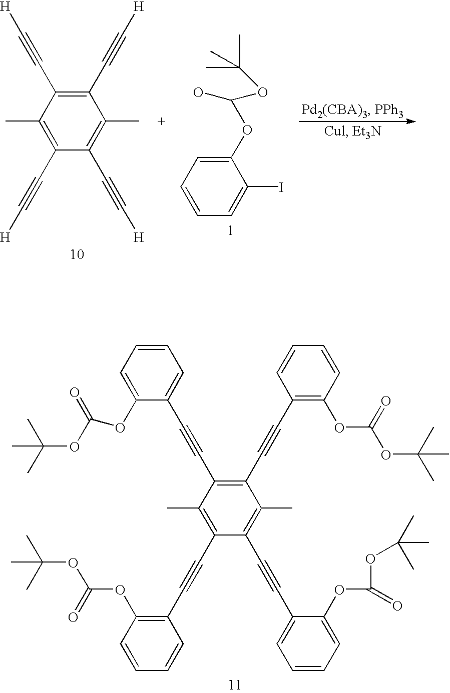

The phenylacetylene compound 10 (590 mg, 2.9×10⁻³ moles), the aryliodide compound 1 (5.6 g, 1.7×10⁻² moles), and triphenylphosphine (613 mg, 2.3×10⁻² moles) were dried under vacuum and flushed with nitrogen. A mixture of copper(I) iodide (56 mg, 2.9×10⁻⁴ moles) and dry triethylamine (50 ml) were degassed and tris(dibenzylideneacetone)dipalladium (267 mg, 2.9×10⁻⁴ moles) was added. The mixture was degassed twice and then transferred via cannular to the flask containing the other reagents and left to stir at room temperature over the weekend. The mixture was filtered through a short plug of SiO₂ washing with dichloromethane before purification by flash chromatography (SiO₂, dichloromethane:cyclohexane:triethylamine (1:1:0.01)). Recrystallisation from dichloromethane/pentane gave compound 11 in the form of an off-white solid (995 mg, 35%).

¹H NMR (300 MHz, CD₂Cl₂) 7.68 (dd, J=1, 8 Hz, 4 H), 7.41 (ddd, J=1 8, 8 Hz, 4 H), 7.28−7.22 (m, 8 H), 2.77 (s, 6 H) 1.44 (s, 36H).

¹³C NMR (75 MHz, CDCl₃) 152.12, 151.65, 140.74, 133.80, 130.26, 126.32, 125.68, 122.67, 117.79, 94,20, 92.26, 84.05, 27.74, 20.19.

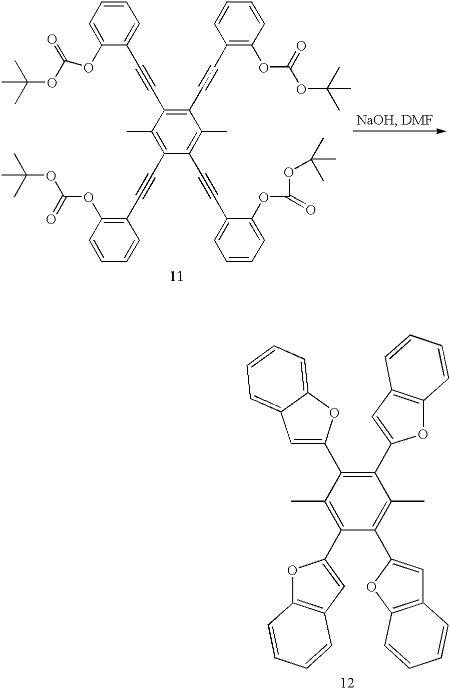

A solution of the phenylacetylene compound 11 (970 mg, 1.0×10⁻³ moles) and sodium hydroxide (200 mg, 5.0×10⁻³ moles) in N,N-dimethylformamide (25 ml) was degassed then heated at reflux overnight. The solvent was distilled under reduced pressure and the residue extracted with methanol. The insoluble material was separated by centrifugation and washed-with methanol (3×20 ml). The resulting solid was dried and then purified by sublimation under reduced pressure (250° C., 10⁻⁴ mbar) to give the desired compound 12, as an off white solid (400 mg, 70%).

¹H NMR (300 MHz, CDCl₃) 7.45−7.42 (m, 8 H), 7.24 (ddd, J=1, 8, 8 Hz, 4 H), 7.15 (ddd, J=1, 8, 8 Hz, 4 H), 6.52 (s, 4 H), 2.18 (s, 6 H).

¹³C NMR (75 MHz, CDCl₃) 154.92, 153.71, 137.87, 134.03, 128.63, 124.22, 122.83, 121.29, 111.33, 106.82, 18.89.

### EXAMPLE 4

### Preparation of 1,3,5-tribenzofuranyl-2,4,6-trimethylbenzene

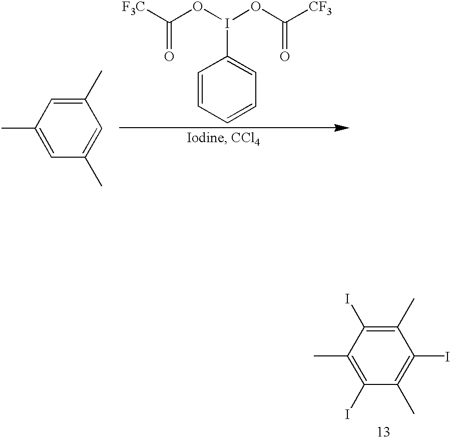

Following the general procedure described in Tett. Lett., 1997, 1485, mesitylene (2.40 g, 2.0×10⁻² moles) was added dropwise to a suspension of [bis(trifluoroacetoxy)iodo]benzene (15.5 g, 3.6×10⁻² moles) and iodine (7.61 g, 3.0×10⁻² moles) in carbon tetrachloride (30 ml). After stirring at room temperature for 1 hour, the iodine colour disappeared and a thick precipitate formed. The precipitate was filtered off and washed with hexanes, recrystallisation from toluene gave compound 13 in the form of white needles (8.0 g, 80%).

¹H NMR (300 MHz, CDCl₃) 3.02 (s, 9 H).

¹³C NMR (75 MHz, CDCl₃) 144.34, 101.39, 39.77.

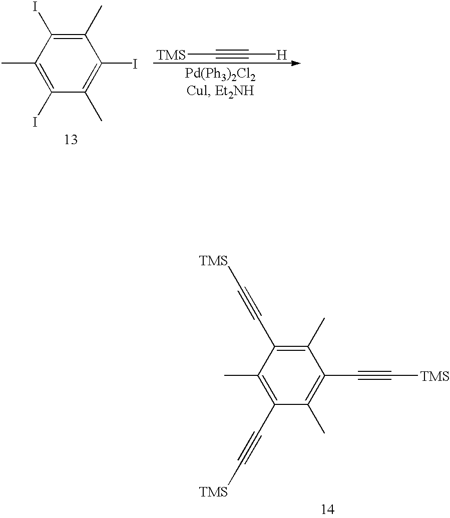

Following the general procedure disclosed in Journal of Organometallic Chemistry, 569, 1998, 195, a mixture of 1,3,5-triiodomesitylene (3.0 g, 6.0×10⁻³ moles), copper(I) iodide (34 mg, 1.8×10⁻⁴ moles) and diethylamine (50 ml) was thoroughly degassed. Dichlorobis(triphenylphosphine)palladium (253 mg, 3.6×10⁻⁴) was added and, after a further degassing, (trimethylsilyl)acetylene (3.54 g, 3.6×10⁻³ moles) was added. The mixture was then briefly degassed and left to stir at room temperature for 6 days during which time a precipitate formed. The mixture was filtered and the precipitate was washed with hexanes. After removing the solvents under reduced pressure, the resulting oil was purified by flash chromatography (SiO₂, hexanes) Recrystallisation from ethanol gave white crystals of compound 14 (1.5 g, 61%).

¹H NMR (300 MHz, CDCl₃) 2.56 (s, 9 H), 0.26 (s, 27 H).

¹³C NMR (75 MHz, CDCl₃) 141.01, 125.51, 104.51, 102.30, 19.82, 0.25.

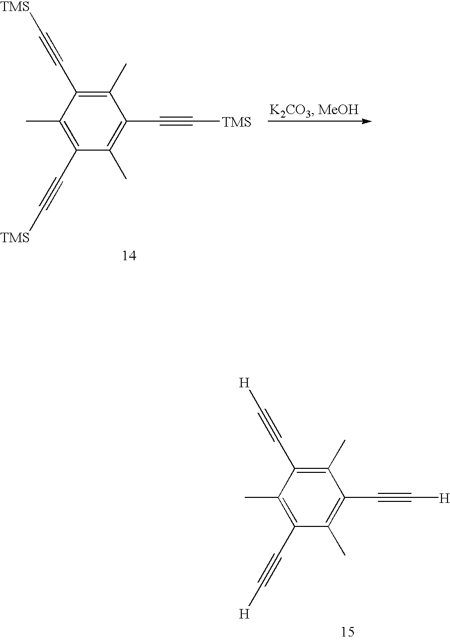

Following the general procedure described in Journal of Organometallic Chemistry, 569, 1998, 195, a suspension of trimethylsilyl protected acetylene 14 (1.5 g, 3.7×10⁻³ moles) and potassium carbonate (868 mg, 6.3×10⁻³ moles) in methanol (50 ml) was heated at 60° C. for three days during which time the mixture became homogeneous. The methanol was removed under reduced pressure and the residue was extracted with benzene (4×20 ml). After removal of the benzene, the product was purified by sublimation (50° C., 10⁻⁴ mbar) to give compound 15 in the form of a purple solid (650 mg, 94%).

¹H NMR (300 MHz, CDCl₃) 3.50 (s, 3 H), 2.62 (s, 9 H).

¹³C NMR (75 MHz, CDCl₃) 144.10, 120.50, 85.49, 80.80, 20.32.

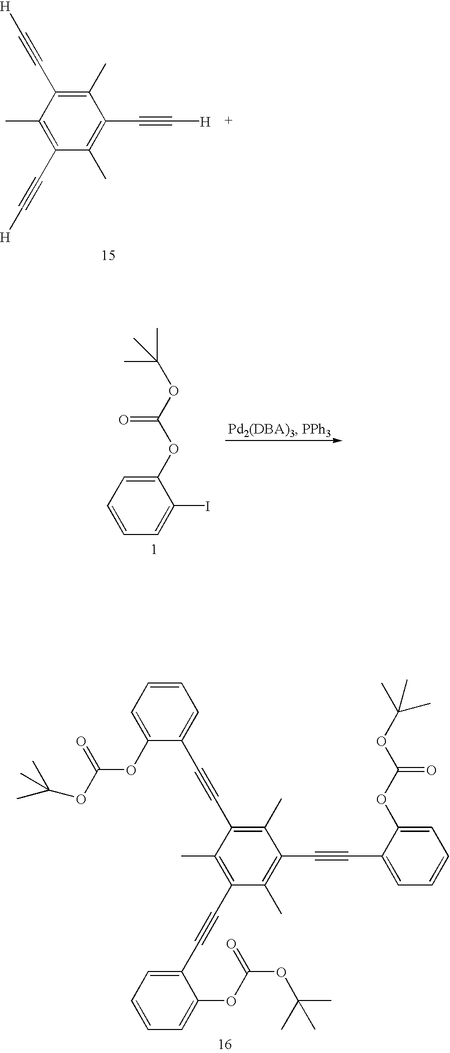

A mixture of triphenylphosphine (525 mg, 2.0×10⁻³ moles), 1.3.5-triethynylmesitylene 15 (650 mg. 3.4×10⁻³ moles) and the aryliodide compound 1 (4.9 g, 1.52×10⁻² moles) were dried under vacuum and flushed with nitrogen. Copper(I) iodide (48 mg, 2.5×10⁻⁴ moles) was dissolved in triethylamine and degassed, then tris(dibenzylideneacetone)dipalladium (232 mg, 2.5×10⁻⁴ moles) was added. This mixture was degassed once more, and then transferred to the flask containing the other ingredients. The reaction was stirred at room temperature for three days during which time a thick precipitate formed. The mixture was dissolved in dichloromethane, washed with water, dried (MgSO₄) and evaporated. The resulting oil was purified by flash chromatography (SiO₂, hexanes:dichloromethane:triethylamine (1:1:0.01) to give compound 16 in the form of a clear oil (2.4 g, 92%).

¹H NMR (300 MHz, CDCl₃) 7.61 (dd, J=1, 8 Hz, 3 H), 7.39 (ddd, J=1 8, 8 Hz, 3 H), 7.30−7.23 (m, 6 H), 2.78 (s, 9 H), 1.51 (s, 27 H).

¹³C NMR (75 MHz, CDCl₃) 151.62, 151.50, 142.79, 133.10, 129.64, 126.13, 122.45, 121.35, 117.78, 92,30, 91.80, 83.93, 27.82, 20.61.

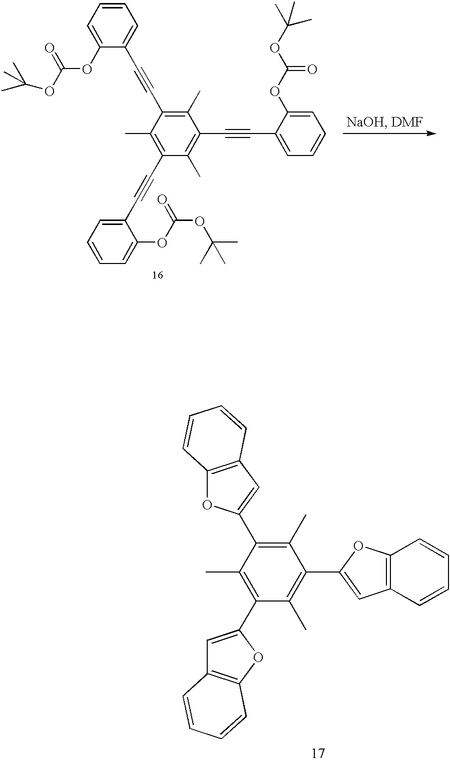

The phenylacetylene compound 16 (2.4 g, 3.1×10⁻³ moles) and sodium hydroxide (600 mg, 1.5×10⁻² moles) in N,N-dimethylformamide (50 ml) was degassed then heated at reflux overnight. The solvent was distilled under reduced pressure and the residue extracted with methanol. The insoluble material was separated by centrifugation and washed with methanol (3×20 ml). The resulting solid was dried and then purified by sublimation (250° C., 10⁻⁴ mbar) to give compound 17 in the form of a bright white solid (1.1 g, 76%).

¹H NMR (300 MHz, CDCl₃) 7.64 (dd, J=1, 8, 3 H), 7.54 (ddd, J=1, 1, 8 Hz, 3 H), 7.32 (ddd, J=1, 8, 8 Hz, 3 H), 7.28 (ddd, J=1, 8, 8, 3H) 6.73 (d, J=1, 3 H), 2.13 (s, 9 H).

¹³C NMR (75 MHz, CDCl₃) 155.06, 154.53, 141.21, 129.83, 128.87, 124.23, 123.03, 121.13, 111.54, 106.84, 19.15.

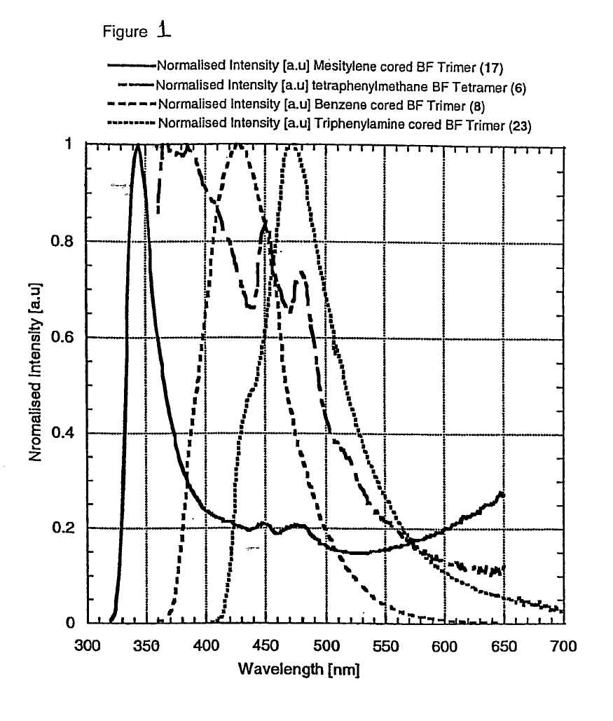

The accompanying FIG. 1 gives an indication of the PL spectrum of an evaporated thin film of benzofuran compound 6 above and the EL spectrum obtained from a single layer device formed of ITO/PEDOT:PSS/Benzofuran/LiF/Al (>50 cdm² at 50 mAcm⁻² 11 V)

### EXAMPLE 5

### Preparation of tris(4-(6-methyl benzofuranyl)phenyl)amine

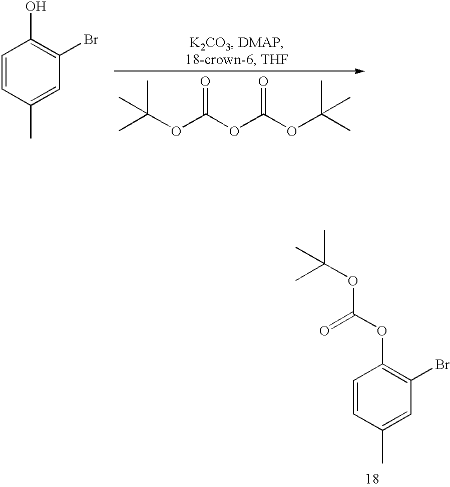

Di-tert-butyl dicarbonate (13 g, 5.96×10⁻² moles) was slowly added to a mixture of 2-bromo-4-methylphenol (10 g, 5.35×10⁻² moles), potassium carbonate (11 g, 7.96×10⁻² moles), Dimethylaminopyridine (catalytic amount) and 18-crown-6 (catalytic amount) in dry tetrahydrofuran (80 ml). The reaction was then left to stir for 2 hours at room temperature. The mixture was then quenched by the addition of brine (200 ml). The product was extracted with diethyl ether (250 ml), the organic layers separated and washed with brine (200 ml). The organic fractions were dried over magnesium sulphate, filtered and evaporated under reduced pressure. The resulting yellow oil was then purified by flash chromatography ( SiO₂, hexanes:dichloromethane (4:1)) and recrystallised from pentane to produce 18 a white, crystalline solid (12.7 g, 83%).

¹H NMR (300 MHz, CDCl₃, 25° C.) δ=1.57 (s, 9 H), 2.34 (s, 3 H), 7.08 (d, J=8.2 Hz, 1 H), 7.12 (dd, J=1.5, 8.4 Hz, 1 H), 7.42 (m, 1 H).

¹³C NMR (75 MHz, CDCl₃, 25° C.) δ=20.58, 27.64, 84.00, 115.90, 123.00, 129.11, 133.64, 137.49, 146.17, 151.07.

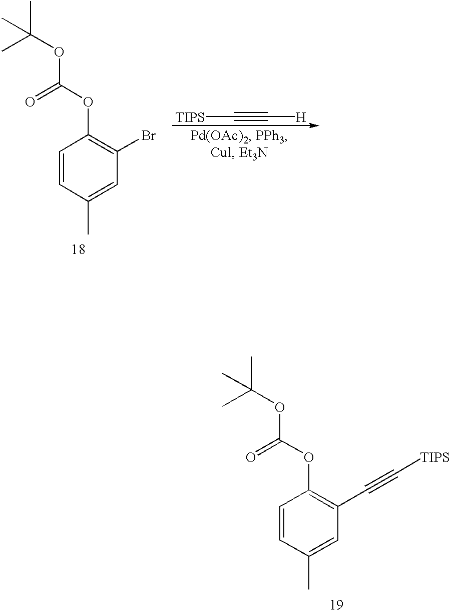

A mixture of arylbromide 18 (10.45 g, 3.64×10⁻² moles), palladium acetate (159 mg, 7.1×10⁻⁴ moles), triphenylphosphine (663 mg, 2.5×10⁻³ moles), copper (I) iodide (69 mg, 3.6×10⁻⁴ moles) and dry triethylamine was degassed (freeze/thaw). (Tri-iso-propylsilyl)acetylene (9.8 g, 5.3×10⁻² moles) was added to the mixture and then heated at 70° C. for 2 hours. In this time a black precipitate had formed which was filtered off and washed with hexane. The yellow filtrate was evaporated and the resulting oil was purified by flash chromatography (SiO₂, hexanes:dichloromethane (3:1)) to give 19 a clear oil (10.5 g, 75%).

¹H NMR (300 MHz, CDCl₃, 25° C.) ∂=1.14 (s, 21 H), 1.54 (s, 9 H), 2.32 (s, 3 H), 7.04 (d, J=8.3 Hz, 1 H), 7.13 (dd, J=2.1, 8.9 Hz, 1 H), 7.32 (d, J=2.0 Hz, 1 H);.

¹³C NMR (75 MHz, CDCl₃, 25° C.) ∂=11.24, 18.67, 20.59, 27.63, 83.26, 95.53, 101.45, 117.01, 121.97, 130.04, 134.00, 135.44, 149.57, 151.65.

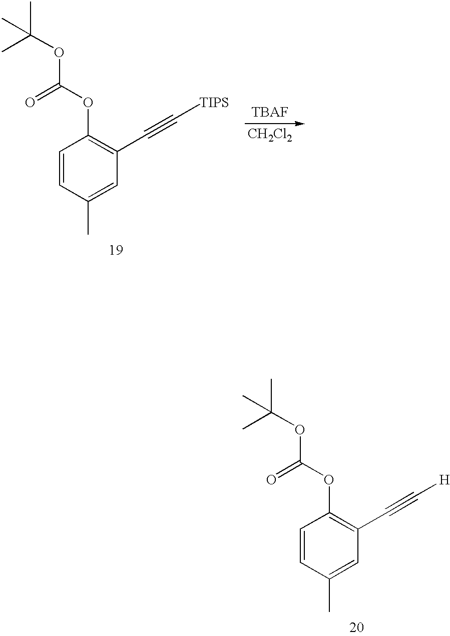

Phenylacetylene 19 (10.4 g, 2.7×10⁻² moles) was dissolved in dichloromethane (500 ml) and tetrabutylammonium fluoride (1 M in THF, 27 ml, 2.7×10⁻² moles) was added. The reaction was stirred at room temperature for 30 minutes, and then quenched by the addition of brine and calcium chloride (100 ml). The product was extracted with dichloromethane (400 ml), the organic fractions dried over magnesium sulphate and the solvent evaporated under reduced pressure. The resulting oil was purified by flash chromatography (SiO₂, hexanes:dichloromethane (3:1)) and then distillation (bp.75° C., 0.001 bar). The clear oil formed was then washed out of the distillation apparatus with pentane, and solidified on evaporation of the solvent to give 20 a white crystalline solid (3.88 g, 63%)

¹H NMR (300 MHz, CDCl₃, 25° C.) ∂=1.56 (s, 9 H), 2.33 (s, 3 H), 3.24 (s, 1 H), 7.05 (d, J=8.3 Hz, 1 H), 7.17 (dd, J1.8, 9.1 Hz, 1 H), 7.34 (m, 1 H).

¹³C NMR (75 MHz, CDCl₃, 25° C.) ∂=20.62, 27.65, 78.50, 81.85, 83.71, 115.90, 121.69, 130.72, 133.91, 135.65, 150.15, 151.37.

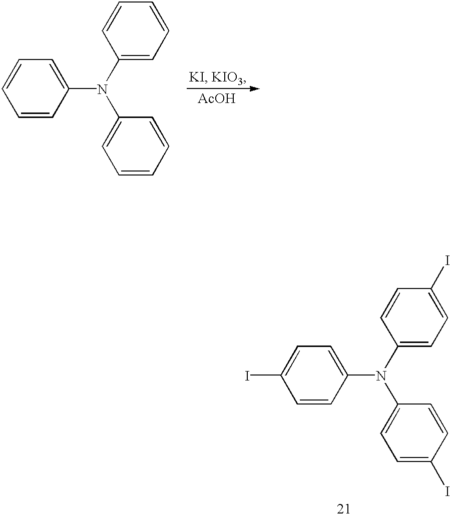

Potassium iodide (5.08 g, 3.06×10⁻² moles) was added portionwise to a mixture of triphenylamine (2.5 g, 1.02×10⁻² moles) and potassium iodate (6.55 g, 3.06×10⁻² moles) in acetic acid (50 ml) and heated to 80° C. overnight. The mixture was then quenched with sodium thiosulphate solution (100 ml), neutralised with sodium hydroxide and extracted with dichloromethane (800 ml). The resulting yellow oil was then purified by flash chromatography ( SiO₂, hexanes:dichloromethane (1:1)), and recrystallised from cyclohexane to give a pale green, crystalline solid (3.28 g, 52%)

¹H NMR (300 MHz, CDCl₃, 25° C.) ∂=6.82 (dd, J=2.8, 8.8 Hz, 6 H), 7.54 (dd, J=2.9, 8.7 Hz, 6 H).

¹³C NMR (75 MHz, CDCl₃, 25° C.) ∂=86.54, 126.03, 138.43, 146.55.

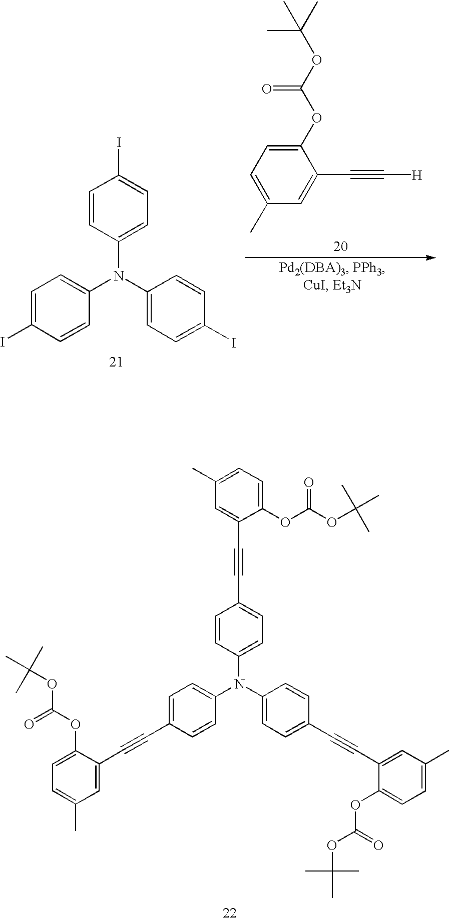

A mixture of tris(4-iodophenyl)-amine 21 (0.84 g, 1.34×10⁻³ moles), phenylacetylene 20 (1.25 g, 5.36×10⁻³ moles) and triphenylphosphine (281 mg, 1.07×10⁻³ moles) was dried under vacuum and flushed with nitrogen. Added to this was a degassed (freeze-thaw) mixture of copper iodide (26 mg, 1.34×10⁻⁵ moles), tris(dibenzylideneacetone)dipalladium (123 mg, 1.34×10⁻⁵ moles) and triethylamine (50 ml). The resulting mixture was then placed under nitrogen and heated at 60° C. overnight. The mixture was then filtered through a short plug of SiO-₂ eluting with dichloromethane before purification by flash chromatography (SiO₂, dichloromethane:hexane:triethylamine (3:1:0.01)). The yellow solid formed was recrystallised from pentane/dichloromethane to give a yellow solid. Yield: 1.05 g, 84%.

¹H NMR (300 MHz, CDCl₃, 25° C,) ∂=1.54 (s, 27 H), 2.35 (s, 9 H), 7.07 (m, 9 H), 7.15 (dd, J=1.9, 8.3 Hz), 7.37 (d, J=1.7 Hz, 3 H), 7.43 (d, J=8.6 Hz, 6 H).

¹³C NMR (75 MHz, CDCl₃, 25° C.) ∂=20.71, 27.70, 83.51, 84.18, 93.90, 117.18, 117.86, 121.70, 123.91, 130.06, 132.84, 133.21, 135.71, 146.74, 149.36, 151.56.

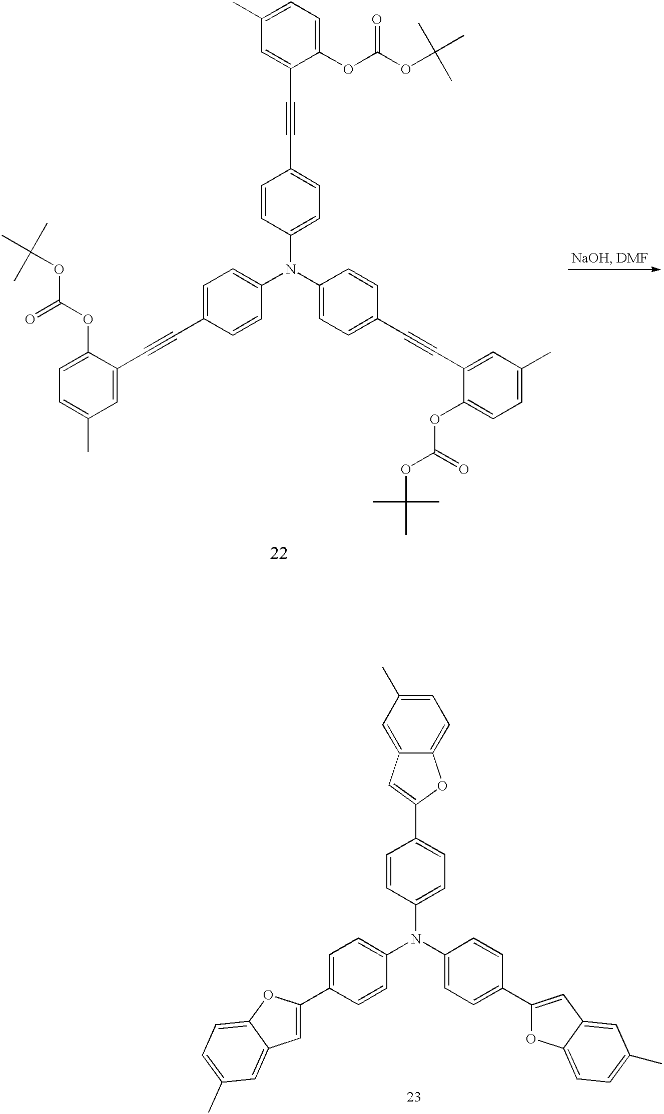

A suspension of phenylacetylene compound 22 (1 g, 1.07×10⁻³ moles) and sodium hydroxide (260 mg, 6.42×10⁻³ moles) in N,N-dimethylformamide (50 ml) was degassed then heated at reflux overnight. The mixture was then cooled and methanol(100 ml) was added, with the insoluble material separated by centrifugation and then washed with methanol. The resulting yellow solid was purified by flash chromatography (SiO₂, dichloromethane:hexane:triethylamine (3:1:0.01)) and then recrystallised from cyclohexane and toluene to give 23 a bright yellow solid. (503 mg, 74%).

¹H NMR (300 MHz, CDCl₃, 25° C.) ∂=2.46 (s, 9 H), 6.90 (s, 3 H), 7.09 (d, J8.4 Hz, 3 H), 7.23 (d, J=8.5 Hz, 6 H), 7.37 (s, 3 H), 7.40 (d, J=8.4 Hz, 3 H), 7.78 (d, J8.5 Hz, 6 H).

¹³C NMR (75 MHz, CDCl₃, 25° C.) ∂=21.35, 100.30, 110.54, 120.55, 124.35, 125.28, 125.59, 126.00, 129.46, 132.33, 147.04, 153.24, 155.75.

### EXAMPLE 6

### Preparation of tris(4-(5-methyl benzofuranyl)phenyl)amine

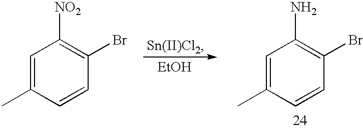

Tin(II)chloride dihydrate (62.3 g, 2.76×10⁻¹ moles) was added portionwise to a mixture of 4-bromo-3-nitrotoluene (20 g, 9.2×10⁻² moles) and ethanol (250 ml) and the mixture was heated to 70° C. for 45 minutes. The mixture was cooled, washed with water, neutralised with sodium hydroxide and the product extracted with dichloromethane (1 l). The resulting brown oil was purified by flash chromatography (SiO₂, hexanes:dichloromethane (1:1)) to give 24 a yellow oil (9.15 g, 53%)

¹H NMR (300 MHz, CDCl₃, 25° C.) ∂=2.24 (s, 3 H), 4.01 (s, 2 H), 6.45 (dd, J=1.9, 8.1 Hz, 1 H), 6.60 (d, J=1.4 Hz, 1 H), 7.28 (d, J=8.1 Hz, 1 H).

¹³C NMR (75 MHz, CDCl₃, 25° C.) ∂=21.18, 105.95, 116.34, 120.33, 132.02, 138.23, 143.62.

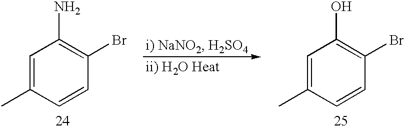

Using the procedure described in Organic Synthesis, CV 3, 130, 2-bromo-5-methylaniline 24 (10 g, 5.37×10⁻² moles) was added to dilute sulphuric acid (9.7 ml in 30 ml of water), stirred and cooled to 15° C. Ice (25 g) was then added to mixture and left to cool until the temperature of the mixture fell below 5° C., upon which sodium nitrite (3.7 g, 5.37×10⁻² moles) was added dropwise and stirred. To this mixture cold water (50 ml) and cracked ice (50 g) was then added with the resulting mixture being placed in an ice bath for 20 minutes. A mixture of sodium sulphate (30 g) and dilute sulphuric acid (100 ml) was set up for steam distillation and heated at 130-135° C. The diazonium solution was added portionwise to this mixture at the same rate as the distillate collected. The distillate was then extracted with diethyl ether (150 ml) washed with water (75 ml) and sodium carbonate solution (75 ml). The product was extracted from the ether layer with sodium hydroxide solution (100 ml) and then acidified using hydrochloric acid (30 ml). The product was then extracted with diethyl ether (150 ml), dried over magnesium sulphate (50 g) and evaporated under reduced pressure. The product was then purified by flash chromatography (SiO₂, hexanes:dichloromethane (2:1)) to give 25 a white crystalline solid. (1.84 g, 18%)

¹H NMR (300 MHz, CDCl₃, 25° C.) ∂=2.30 (s, 3 H), 5.42 (s, 1 H), 6.64 (dd, J=1.5, 8.1 Hz, 1 H), 6.86 (d, J=1.3 Hz, 1 H), 7.33 (d, J=8.2 Hz, 1 H).

¹³C NMR (75 MHz, CDCl₃, 25° C.) ∂=21.02, 106.80, 116.67, 122.72, 131.47, 139.54, 151.86.

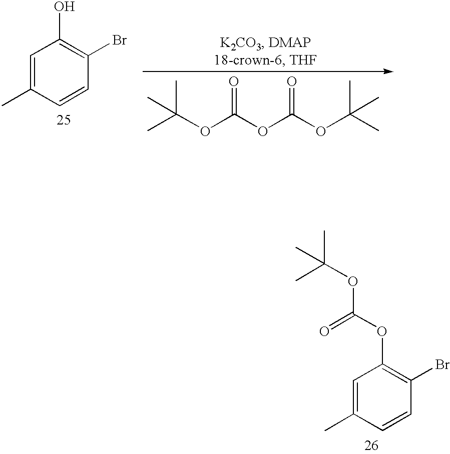

Di-tert-butyl dicarbonate (16.8 g, 7.69×10⁻² moles) was slowly added to a mixture of 2-bromo-5-methylphenol 25 (11.5 g, 6.15×10⁻² moles), potassium carbonate (12.75 g, 9.23×10⁻² moles), Dimethylaminopyridine (catalytic amount) and 18-crown-6 (catalytic amount) in dry tetrahydrofuran (150 ml). The reaction was then left to stir for 2 hours at room temperature. The mixture was then quenched by the addition of brine (200 ml). The product was extracted with diethyl ether (250 ml), the organic layers separated and washed with brine (200 ml). The organic fractions were dried over magnesium sulphate, filtered and evaporated under reduced pressure. The resulting yellow oil was then purified by flash chromatography (SiO₂, hexanes:dichloromethane (4:1)) and recrystallised from pentane to produce 26 a white, crystalline solid. (15.5 g, 88%).

¹H NMR (300 MHz, CDCl₃, 25° C.) ∂=1.58 (s, 9 H), 2.33 (s, 3 H), 6.94 (dd, J=1.7, 8.1 Hz, 1 H), 7.03 (d, J=1.8 Hz, 1 H), 7.47 (d, J=8.1 Hz, 1 H).

¹³C NMR (75 MHz, CDCl₃, 25° C.) ∂=20.90, 27.63, 84.07, 112.82, 124.06, 128.24, 132.85, 139.01, 148.03, 150.99.

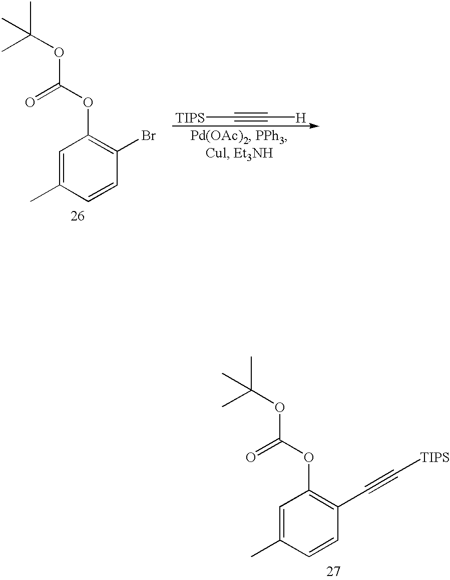

Arylbromide 26 (5.5 g, 1.92×10⁻²moles) was added to a mixture of palladium acetate (87 mg, 3.84×10⁻⁴ moles), triphenylphosphine (404 mg, 1.54×10⁻³ moles), copper(I)iodide (37 mg, 1.92×10⁻⁴ moles) and dry di-iso-propylamine (60 ml) and degassed (freeze/thaw). (Tri-iso-propylsilyl)acetylene (5.27 g, 2.89×10⁻² moles) was then added to the mixture and heated at 80° C. for 4 hours, after which tris(dibenzylideneacetone)dipalladium (176 mg, 1.92×10⁻⁴ moles) was added. The mixture was then heated at 80° C. overnight. In this time a black precipitate had formed which was filtered off and washed with hexane. The yellow filtrate was evaporated under reduced pressure and the resulting oil was purified by flash chromatography (SiO-₂, hexanes:dichloromethane (3:1)) to give a clear oil 27 (6.1 g, 82%)

¹H NMR (300 MHz, CDCl₃, 25° C.) ∂=1.14 (s, 21 H), 1.55 (s, 9 H), 2.36 (s, 3 H), 6.99 (m, 2 H), 7.40 (d, J7.14 Hz, 1 H).

¹³C NMR (75 MHz, CDCl₃, 25° C.) ∂=11.25, 18.69, 21.41, 27.66, 83.35, 95.08, 101.40, 114.51, 122.82, 126.61, 133.46, 140.07, 151.51, 151.59.

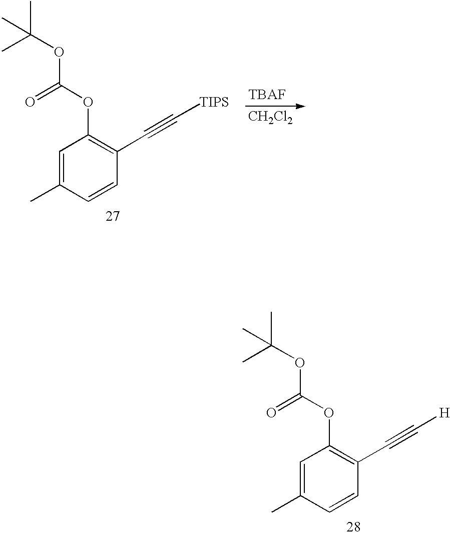

Phenylacetylene compounds 27 (14.2 g, 3.65×10⁻² moles) was dissolved in dichloromethane (270 ml) and tetrabutylammonium fluoride (1 M in THF, 37 ml, 3.7×10⁻¹ moles) was added. The reaction was stirred at room temperature for 30 minutes, and then quenched by the addition of brine and calcium chloride (100 ml). The product was extracted with dichloromethane (400 ml), the organic fractions dried over magnesium sulphate and the solvent evaporated. The resulting solid was purified by flash chromatography (SiO₂, hexanes:dichloromethane (2:1)), distillation (bp.75° C., 0.001 bar) and recrystallised from pentane to give 28, a white solid (5.88 g, 69%)

¹H NMR (300 MHz, CDCl₃, 25° C.) ∂=1.57 (s, 9 H), 2.37 (s, 3 H), 3.23 (s, 1 H), 6.99 (s, 1 H), 7.02 (d, J=8.5 Hz, 1 H), 7.42 (d, J=7.8 Hz, 1 H).

¹³C NMR (75 MHz, CDCl₃, 25° C.) ∂=21.42, 27.65, 78.45, 81.57, 83.79, 113.25, 122.60, 126.74, 133.27, 140.88, 151.31, 152.12.

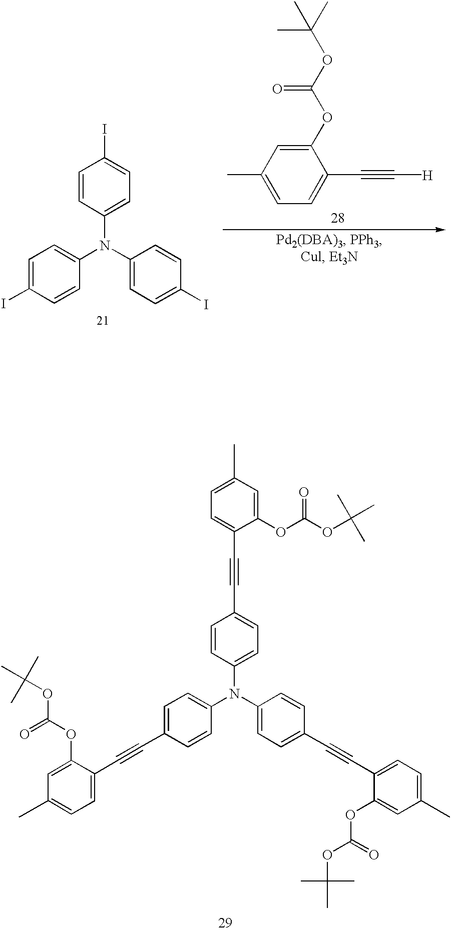

A mixture of tris(4-iodophenyl)amine (21) (0.84 g, 1.34×10⁻³ moles) phenylacetylene compound 28 (1.25 g, 5.36×10⁻³ moles) and triphenylphosphine (281 mg, 1.07×10⁻³ moles) was dried under vacuum and flushed with nitrogen. Added to this was a degassed (freeze-thaw) mixture of copper iodide (26 mg, 1.34×10⁻⁵ moles), tris(dibenzylideneacetone)dipalladium (123 mg, 1.34×10⁻⁵ moles) and triethylamine (50 ml). The resulting mixture was then placed under nitrogen and heated at 65° C. overnight. The mixture was then filtered through a short plug of SiO-₂ eluting with dichloromethane before purification by flash chromatography (SiO₂, dichloromethane:hexane:triethylamine (2:1:0.01)). The resulting solid was then recrystallised from cyclohexane to give 29 a yellow solid (980 mg, 78%)

¹H NMR (300 MHz, CDCl₃, 27° C.) ∂=1.54 (s, 27 H), 2.38 (s, 3 H), 7.01 (m, 3 H), 7.05 (d, J=9.3 Hz, 3 H), 7.06 (d, J=8.7 Hz, 6 H), 7.42 (d, J=8.6 Hz, 6 H), 7.44 (d, J=7.7 Hz, 3 H).

¹³C NMR (75 MHz, CDCl₃, 27° C.) ∂=21.44, 27.71, 83.58, 84.12, 93.59, 114.55, 117.94, 122.64, 123.89, 126.86, 132.57, 132.77, 140.14, 146.67, 151.32, 151.51.

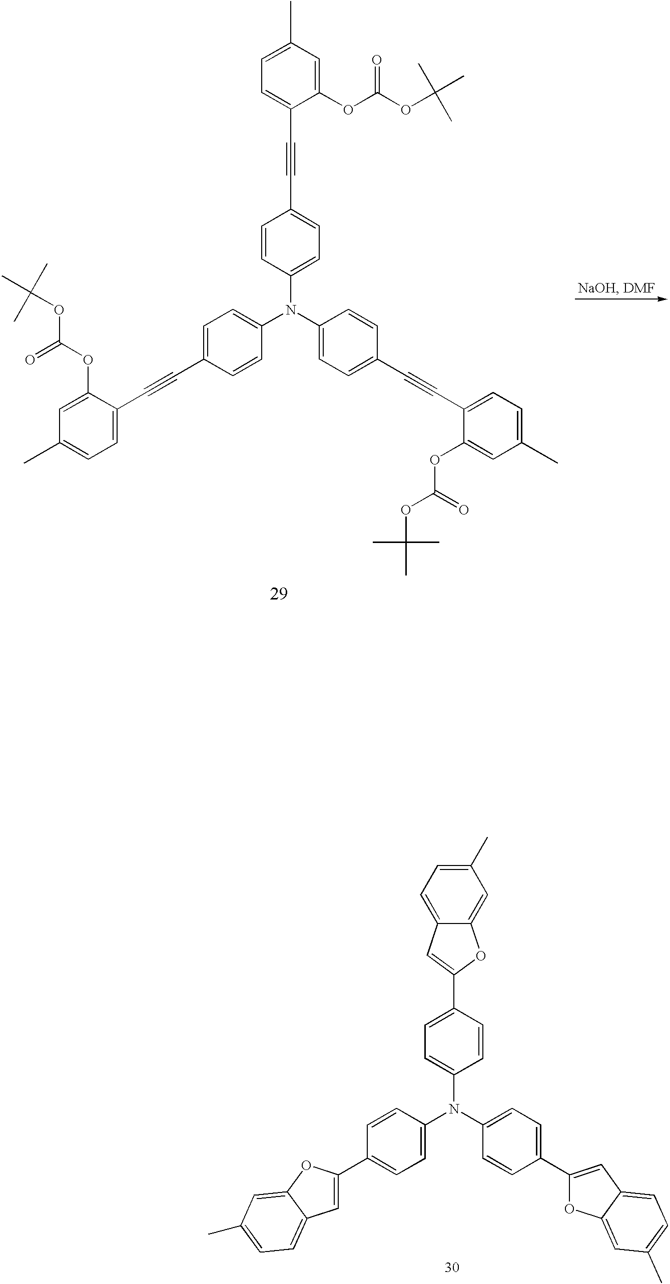

A suspension of phenyl acetylene compound 29 (0.95 g, 1.01×10⁻³ moles) and sodium hydroxide (240 mg, 6.06×10⁻³ moles) in N,N-dimethylformamide (50 ml) was degassed then heated at reflux overnight. The mixture was then cooled and quenched with methanol, with the insoluble material separated by centrifugation. The resulting yellow solid was purified by flash chromatography (SiO₂, dichloromethane:hexane:triethylamine (3:1:0.01)) and then recrystallised from cyclohexane and toluene to give 30 a bright yellow solid (400 mg, 62%).

¹H NMR (300 MHz, CDCl₃, 25° C.) ∂=2.50 (s, 9 H), 6.92 (s, 3 H), 7.07 (dd, J=1.0, 7.8 Hz, 3 H) 7.23 (d, J=8.7 Hz, 6 H), 7.33 (s, 3 H), 7.45 (d, J=7.9 Hz, 3 H), 7.77 (d, J=8.7 Hz, 6 H).

¹³C NMR (75 MHz, CDCl₃, 25° C.) ∂=21.76, 100.39, 111.34, 120.16, 124.30, 124.34, 125.62, 125.88, 126.83, 134.32, 146.95, 155.12, 155.23.

### EXAMPLE 7

### Preparation of 2,5-Bis-[3,5-bis-(5-methyl-benzofuran-2-yl)-phenyl]-[1,3,4]oxadiazole

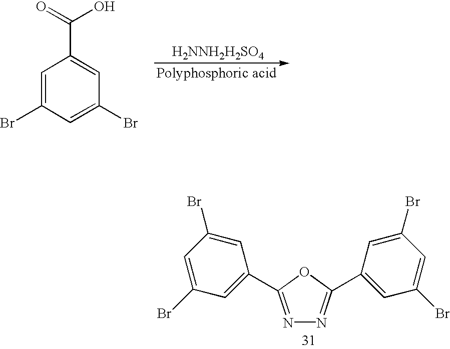

Hydrazine sulphate (0.23 g, 1.79×10⁻³ moles) was added to a mixture of 3,5-dibromobenzoic acid (1 g, 3.57×10⁻³ moles) and polyphosphoric acid (5 g), and the mixture heated to 130° C. overnight. The resulting mixture was cooled, neutralised with sodium hydroxide solution (50 ml) and stirred for 15 minutes. The product was extracted with dichloromethane (100 ml), dried over magnesium sulphate and purified by flash chromatography (SiO2, dichloromethane:hexanes (2:1)) to give 31, a white solid (0.7 g, 73%).

¹H NMR (300 MHz, CDCl₃, 25° C.) δ=8.24 (d, J=1.7 Hz, 4H), 7.89 (t, J=1.7 Hz, 2H).

¹³C NMR (75 MHz, CDCl₃, 25° C.) δ=163.15, 137.92, 128.96, 126.84, 124.25

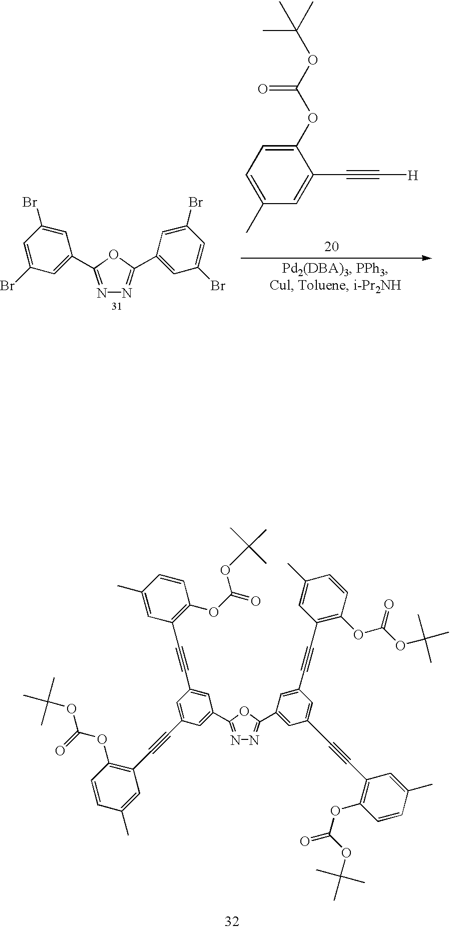

A mixture of oxadiazole 31 (0.5 g, 9.29×10⁻⁴ moles), phenylacetylene 20 (1.29 g, 5.57×10⁻³ moles), triphenylphosphine (195 mg, 7.43×10⁻⁴ moles), copper iodide (18 mg, 9.29×10⁻⁵ moles) and tris(dibenzylideneacetone)dipalladium (85 mg, 9.29×10⁻⁵ moles) was dried under vacuum and flushed with nitrogen. Added to this was a degassed (freeze-thaw) mixture of toluene (30 ml) and di-iso-propylamine (6 ml). The resulting mixture was then placed under nitrogen and heated at 80° C. overnight. The mixture was then filtered and purified by flash chromatography (SiO₂, dichloromethane:hexane:triethylamine (3:2:0.01)). The crude solid formed was recrystallised from cyclohexane/toluene to give 32 a white solid (1.02 g, 96%)

¹H NMR (300 MHz, CDCl₃): 8.25 (d, J=1.6 Hz, 4H), 7.86 (t, J=1.6 Hz, 2H), 7.42 (d, J=2.0 Hz, 4H), 7.22 (dd, J=2.1, 8.5 Hz, 4H), 7.11 (d, J=8.3 Hz, 4H), 2.38 (s, 12H), 1.57 (s, 36 H).

¹³C NMR (75 MHz, CDCl₃): 164.10, 151.98, 150.15, 137.88, 136.27, 133.83, 131.36, 129.57, 125.26, 124.87, 122.33, 116.75, 92.02, 87.00, 84.30, 28.11, 21.12.

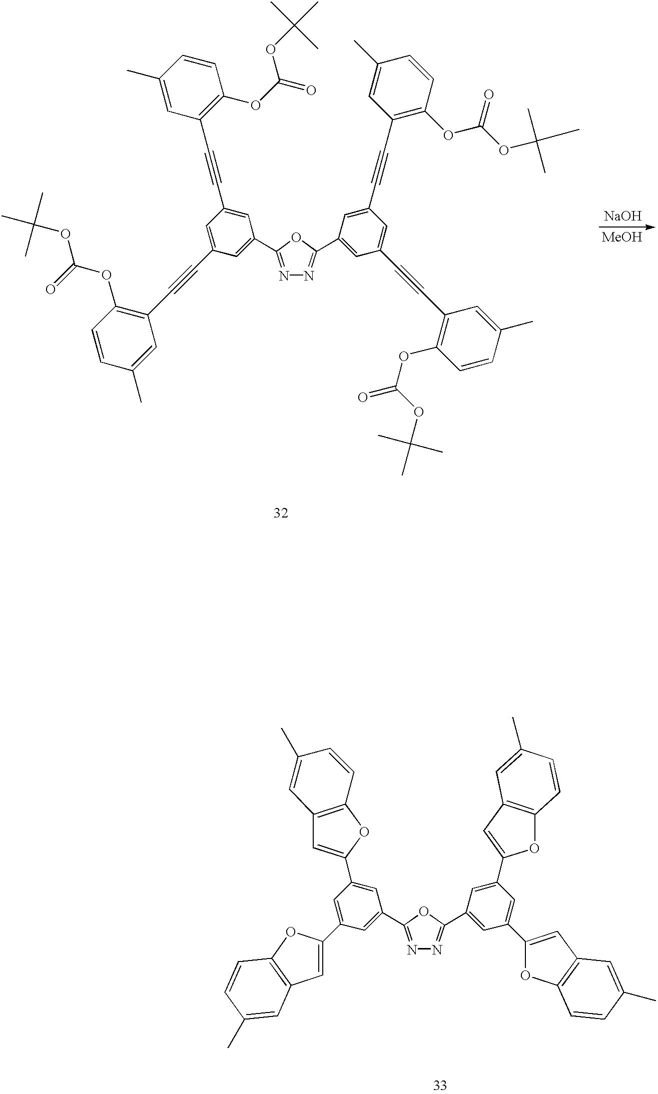

Phenylacetylene 32 (0.3 g, 2.62×10⁻⁴ moles) was placed in a drying oven and heated at 160° C. for 2 hours at 0.1 mbar. After cooling methanol (30 ml) and sodium hydroxide (84 mg, 2.1×10⁻³) were added. The resulting mixture was then degassed and heated at reflux overnight. The mixture was then cooled, with the insoluble material separated by centrifugation and washed with methanol. This gave compound 33, a tan coloured solid (120 mg, 62%).

¹H NMR (300 MHz, CDCl₃): 8.58 (d, J=1.6 Hz, 4H), 8.50 (t, J=1.6 Hz, 2H), 7.52 (d, J=8.4, 4H), 7.45 (s, 4H), 7.26 (s, 4H), 7.19 (dd, J=1.4, 8.5 Hz, 4H), 2.50 (s, 12H).

MS (MALDI-TOF) M/Z 743.32 (MH⁺ calcd m/z 743.25)

### EXAMPLE 8

### Preparation of tris[3-methyl-4(5-methyl-benzofuran-2-yl)phenyl]amine

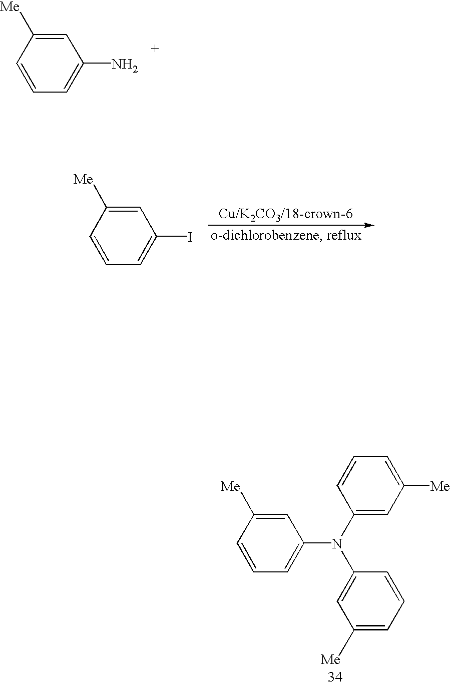

m-Toluidine (0.5 g, 4.66×10⁻³ moles), 3-iodotoluene (2.55 g, 1.17×10⁻² moles), powdered anhydrous potassium carbonate (5.18 g, 3.73×10⁻² moles), electrolytic copper powder (1.19 g, 1.88×10⁻² moles) and 18-crown-6 (0.25 g, 9.4×10⁻⁴ moles) were refluxed in o-dichlorobenzene (9 ml), under nitrogen, overnight. The solution was diluted with dichloromethane (30 ml) and the copper powder and inorganic salts were removed by filtration. The dichloromethane was then removed by distillation under reduced pressure. The dichlorobenzene was removed by distillation under reduced pressure. The product was purified by flash chromatography (SiO₂, cyclohexane increasing polarity to dichloromethane:cyclohexane 1:5) giving compound 34 a white waxy solid (0.54 g, 40%)

¹H NMR (300 MHz, CDCl₃) 7.12 (t, J=8, 3H), 6.9−6.8 (m, 9H), 2.25 (s, 9H).

¹³C NMR (75 MHz, CDCl₃) 147.97, 138.92, 128.88, 124.80, 123.34, 121.38, 21.41.

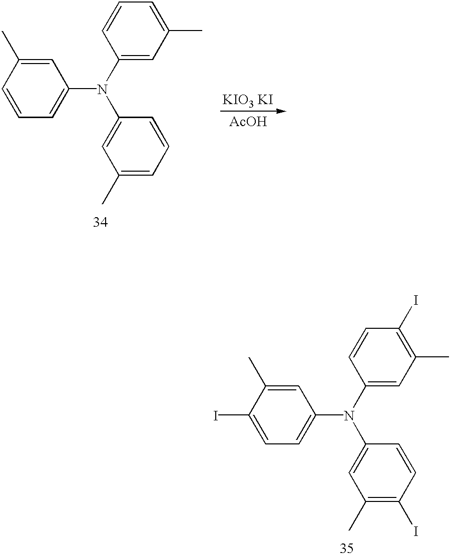

Potassium iodide (0.87 g, 5.22×10⁻³ moles) was added portionwise to a mixture of tris(3-methylphenyl)-amine 34 (0.5 g, 1.74×10−3 moles) and potassium iodate (5.22×10⁻³ moles, 1.12 g) in acetic acid (9 ml) and the mixture was heated to 80° C. over night. The mixture was quenched with sodium thiosulphate solution (2 wt %, 100 ml), neutralised with sodium hydroxide (2 M) and extracted with dichloromethane (200 ml). The product was purified by column chromatography (SiO2, hexanes) and recrystalisation from dichloromethane/methanol to give compound 35 a white solid (0.34 g 30%)

¹H NMR (300 MHz, CDCl₃) 7.63 (d, J=8, 3H), 6.91 (d, J=3, 3H), 6.58 (dd, J=8, J=3, 3H), 2.32 (s, 9H).

¹³C NMR (75 Hz, CDCl₃) 147.30, 142.50, 139.55, 125.414, 123.40, 93.32, 28.07

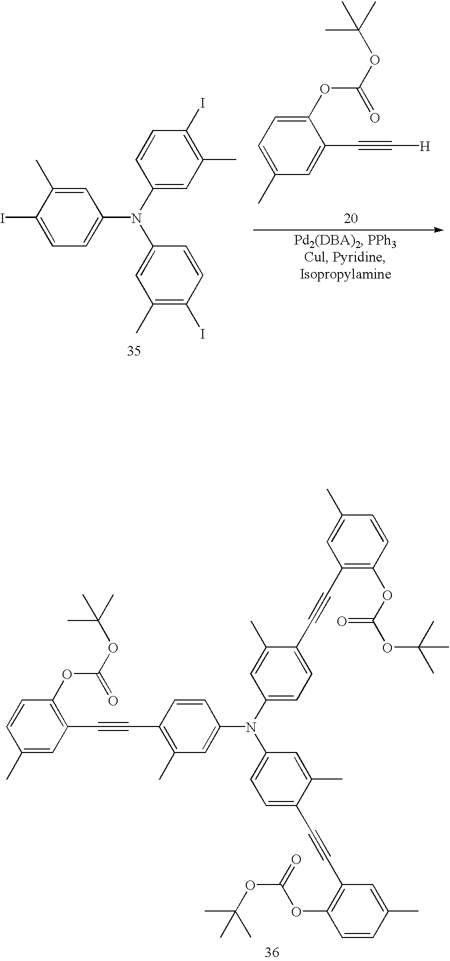

A mixture of tris(4-iodo-3-methylphenyl)-amine 35 (250 mg, 3.76×10⁻⁴ moles), phenylacetylene 20 (350 mg, 1.50×10⁻³ moles) and triphenylphosphine (79 mg, 3.00×10⁻⁴ moles) were dried under vacuum and flushed with nitrogen. A degassed mixture of copper iodide (7.2 mg, 3.76×10⁻⁵ moles,), tris(dibenzylideneacetone)-dipalladium (35 mg, 3.76×10⁻⁵ moles,) and diisopropylamine (14 ml) were added and the resulting mixture was placed under nitrogen and heated at 60° C. overnight. The mixture was diluted with cyclohexane and then filtered. After the solvent was removed the product was purified by flash chromatography (SiO₂, dichloromethane:cyclohexane—1:4). The product was recyrstalised from cyclohexane to give compound 36, a pale yellow crystalline solid (130 mg, 35%).

¹H NMR (300 MHz, CDCl₃) 7.39 (d, J=8, 3H), 7.37 (d, J=2, 3H), 7.14 (dd, J=2, J=8, 3H), 7.08 (d, J=8, 3H), 6.94 (d, J=2, 3H), 6.89 (dd, J=2, J=8, 3H), 2.45 (s, 9H), 2.35 (s, 9H), 1.54 (s, 27H).

¹³C NMR (75 Hz, CDCl₃) 149.20, 146.97, 141.71, 135.62, 133.07, 132.93 129.84, 124.97, 121.80, 121.48, 117.65, 117.34, 93.04, 87.89, 83.42, 27.68, 20.91, 20.70,

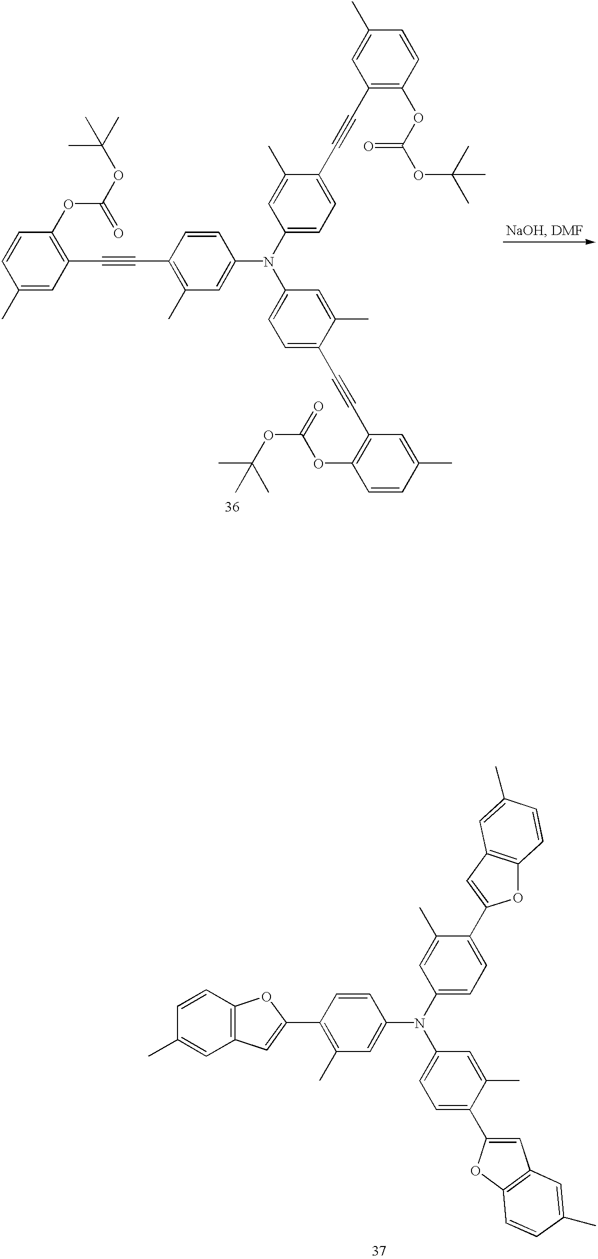

A solution of compound 36 (0.1 g, 1.02×10⁻⁴ moles) and sodium hydroxide (26 mg, 6.13×10⁻⁴ moles) in N,N-dimethylformamide (5 ml) was degassed and heated at reflux overnight. The N,N-dimethylformamide was then removed under reduced pressure and methanol (50 ml) was added to the solid to remove the sodium hydroxide. The solution was allowed to stand and the precipitate that formed was removed by centrifugation after drying a yellow solid 37 reamined (30 mg, 40%)

¹H NMR (300 MHz, CDCl₃) 7.77 (d, J=8, 3H), 7.40−7.38 (m, 6H), 7.10−7.07 (m, 9H), 6.79 (s, 3H), 2.51 (s, 9H), 2.46 (s, 9H),

¹³C NMR (75 Hz, CDCl₃) 155.56, 152.70, 146.99, 137.09, 132.17, 129.44, 129.07, 126.59, 125.28, 124.99, 121.93, 120.57, 110.45, 104.16, 22.10, 21.35.

### EXAMPLE 9

### Preparation of tris[3-(5-methyl-benzofuran-2-yl)phenyl]amine

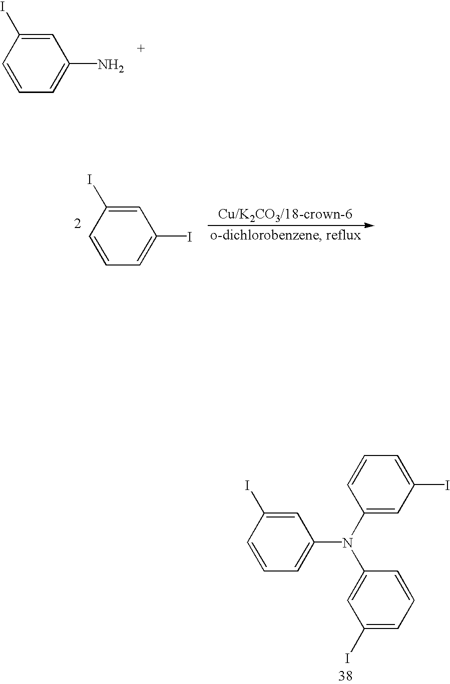

3-Iodoaniline (2.5 g, 1.14×10⁻² moles), 1,3-diiodobenzene (15.04 g, 4.56×10⁻² moles), anhydrous potassium carbonate (12.68 g, 9.17×10⁻² moles), electrolytic copper powder (2.90 g, 4.60×10⁻² moles) and 18-crown-6 (0.61 g, 2.30×10⁻³ moles) were refluxed in o-dichlorobenzene (22 ml), under nitrogen, overnight. After dilution with dichloromethane (50 ml), the copper and inorganic salts were filtered off and the solvents were removed under reduced pressure. The product was purified by flash chromatography (SiO₂, cyclohexane), where the main band was isolated. This was further purified by heating (225° C., 1×10⁻⁵ mbar), to remove volatile components, giving 38 an off white waxy solid (360 mg, 5.1%).

¹H NMR (300 MHz, CDCl₃) 7.42−7.37 (m, 6H), 7.01−6.99 (m, 6H).

¹³C NMR (75 Hz, CDCl₃) 148.01, 132.91, 132.72, 130.94, 123.53, 94.70.

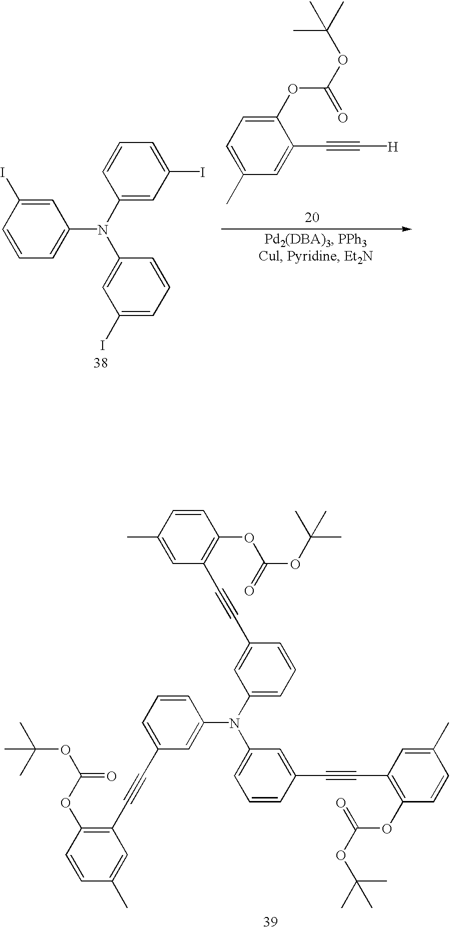

A mixture of tris(3-iodophenyl)amine (360 mg, 5.62×10⁻⁴ moles), phenylacetylene20 (520 mg, 5.62×10⁻³ moles) and triphenylphosphine (117 mg, 4.48×10⁻⁴ moles) were dried under vacuum and flushed with nitrogen. A degassed mixture of copper iodide (11 mg, 5.62×10⁻⁵ moles), tris(dibenzylideneacetone)dipalladium (35 mg, 3.76×10⁻⁵ moles) and diisopropylamine (21 ml) was added and the resulting mixture was placed under nitrogen and heated at 60° C. overnight. The mixture was then filtered. After the solvent was removed, the product was purified by flash chromatography (SiO₂, dichloromethane:cyclohexane:triethylamine—2:1:0.01), to give 39 a yellow solid (250 mg, 48% yield).

¹H NMR (300 MHz, CDCl₃) 7.35 (d, J=2, 3H), 7.26−7.19 (m, 9H), 7.14 (dd, J=2, J=8, 3H), 7.08−7.03 (m, 6H), 2.33 (s, 9H), 1.46 (s, 27H).

¹³C NMR (75 Hz, CDCl₃) 151.50, 149.41, 147.36, 135.68, 133.41, 130.30, 129.54, 127.21, 126.77, 124.71, 124.48, 121.71, 116.97, 93.57, 84.63, 83.59, 27.62, 20.66.

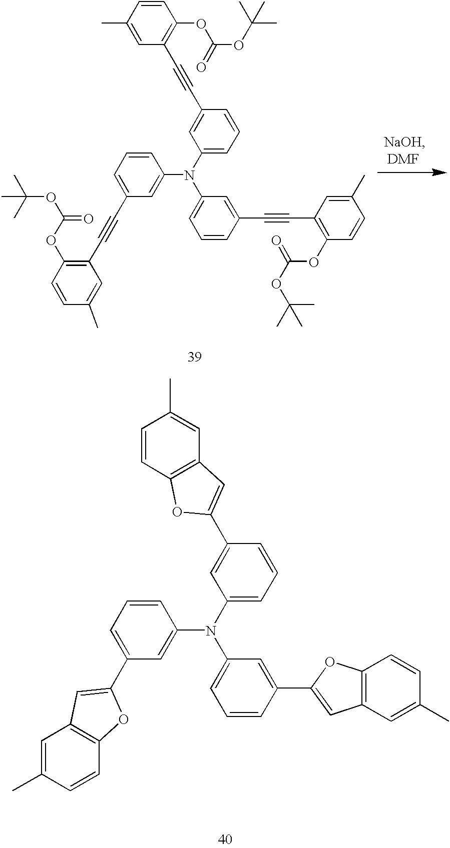

A suspension of compound 39 (210 mg, 2.25×10⁻⁴ moles) and sodium hydroxide (54 mg, 1.35×10⁻³ moles,) in N,N-dimethylformamide (11 ml) was degassed and heated at reflux overnight. The solvent was removed under reduced pressure and methanol (50 ml) was added. The mixture was sonicated in an ultrasound bath for 30 minutes. The solution was allowed to stand and the precipitate was removed by centrifugation, to give 40 an off white solid (120 mg, 84%)

¹H NMR (300 MHz, CDCl₃) 7.68 (t, J=2, 3H), 7.58 (dt, J=1, J=8, 3H), 7.38 (t, J=8, 3H), 7.35 (d, J=8, 3H), 7.32 (t, J=1, 3H), 7.15 (ddd, J=1, J=2, J=8, 3H), 7.06 (dd, J=2, J=8, 3H), 6.89 (d, J=1, 3H), 2.42 (s, 9H).

¹³C (75 Hz, CDCl₃) 155.53, 153.31, 148.00, 132.31, 132.00, 129.93, 129.18, 125.62, 124.45, 120.70, 120.56, 119.75, 110.70, 101.56, 21.31.

Accompanying FIG. 1 shows the EL Spectra of four benzofuran compounds, namely compounds 6, 8, 17 and 23, in an electroluminescent device comprising, in sequence, an ITO electrode, a 50 nm layer of PEDOT:PSS (Baytron P), a 60 nm layer containing the benzofuran compound, a 60 nm OXD-7

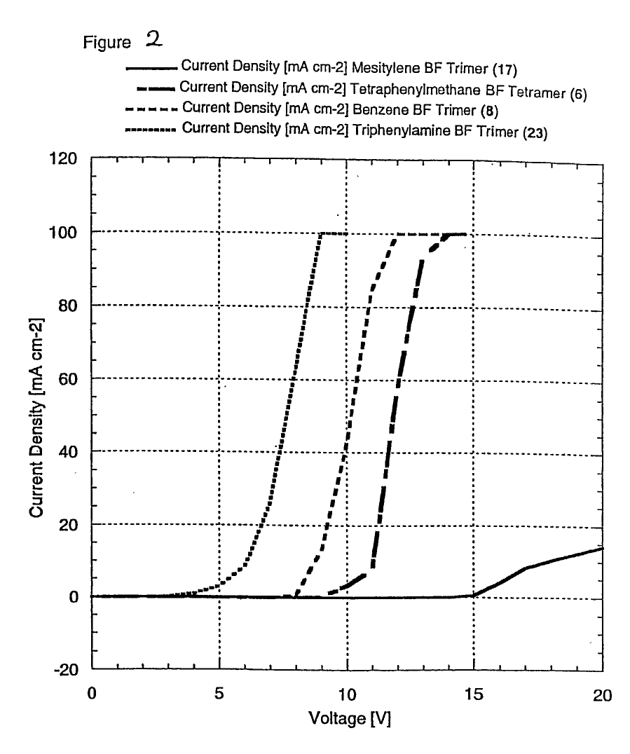

(1,3,4-oxadiazole-2,2′-(1,3-phenylene)bis[5-[4-(1,1-d imethylethyl)phenyl]) electron transport layer, a 1.7 nm insulating layer of LiF and an Aluminium electrode, accompanying FIG. 2 is a graph showing the IV characteristics for the device structures used to generate the data of FIG. 1, and

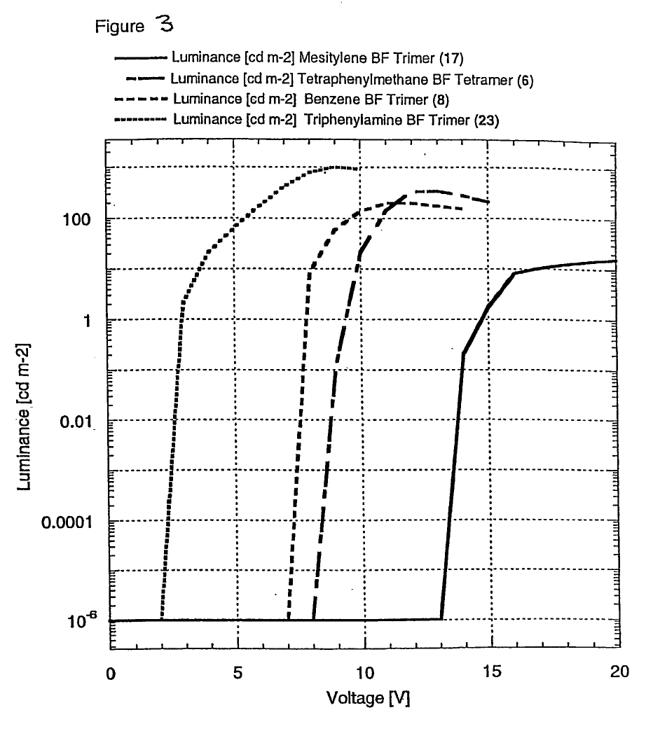

accompanying FIG. 3 is a graph showing the VL characteristics for the devices used to generate the data of FIG. 1.

The data in the above graphs was as a result of measurements carried out under a nitrogen atmosphere.

The table below gives a summary of the performance of the devices above. By altering the core of the materials it is possible to effect both the colour of the emission, the efficiency and lifetime of the devices.

In the above devices the benzofuran compound according to the present invention was used to form the layer. However, it is within the scope of the present invention to incorporate the benzofuran compounds according to the present invention in a host matrix in any appropriate concentration, but preferably at low concentrations (typically less than 5% by weight of the host matrix). Alternatively, it is within the scope of the present invention to use one or more compounds according to the present inventions as host materials and dope them with more emissive dyes for better emission.

| | Turn on | | | | | |
|---|---|---|---|---|---|---|
| | voltage | maximum | Maximum | EL | | |
| | (V) | efficiency | luminance | peak | | |
| Benzofuran | L > 0.1 cd/m² | (Lm/W) | (cd/m²) | (nm) | CIE | Lifetime |
| Benzene | 7 | 0.18@ | 183@ | 430 | 0.15, 0.09 | <60 s |
| 8 | | 7 cd/m² | 4 mA,14 V | | | |
| Mesitylene | 14 | 0.07@ | 54@ | 340 | N/A | <60 s |
| 17 | | 0.12 cd/m² | 2 mA,19.4 V | | | |
| Tetraphenylmethane | 9 | 0.52@ | 520@ | 370 | 0.15, 0.06 | <60 s |
| 6 | | 141 cd/m² | 4 mA,13.1 V | | | |
| Triphenylamine | 3 | 2.3@ | 1000@ | 470 | 0.17, 0.2 | >1.5 hr |
| 23 | | 2 cd/m² | 4 mA,9 V | | | |

What is claimed is:

1. A method of preparing a branched benzofuran compound comprising a core moiety which contains at least one aromatic ring and which has at least three substituted or unsubstituted benzofuran groups covalently linked thereto, said method comprising the steps of
(i) forming an intermediate ethynylene compound in which at least three benzene rings are each linked to the core moiety via an ethynylene bond, and where each benzene ring is substituted at the ortho position (relative to the position of the ethynylene bond) by a blocked carbonyloxy group,
(ii) deblocking the carbonyloxy groups, and
(iii) effecting ring closure by reaction between the deblocked carbonyloxy groups and the adjacent ethynylene bonds to form the furan rings of the benzofuran groups, whereby to produce the branched benzofuran compound.

2. A method as claimed in claim 1, wherein the intermediate ethynylene compound formed in step (i) is prepared by forming ethynylene groups on the core moiety and then reacting each of these with a reactive substituent on a benzene ring which is substituted at the ortho position (relative to the position of the reactive substituent) by a blocked carbonyloxy group.

3. A method as claimed in claim 1, wherein the intermediate ethynylene compound formed in step (i) is prepared by preparing compounds containing a benzene ring substituted with an ethynylene group and a blocked carbonyloxy group which are in the ortho position with respect to each other, and then reacting the ethynylene groups of said compounds with reactive groups on the core moiety so as to link each of the benzene rings with the core moiety via an ethynylene bond.

4. A method as claimed in claim 1, wherein the core moiety is selected from (a) a core moiety having one of the following ring structures:— 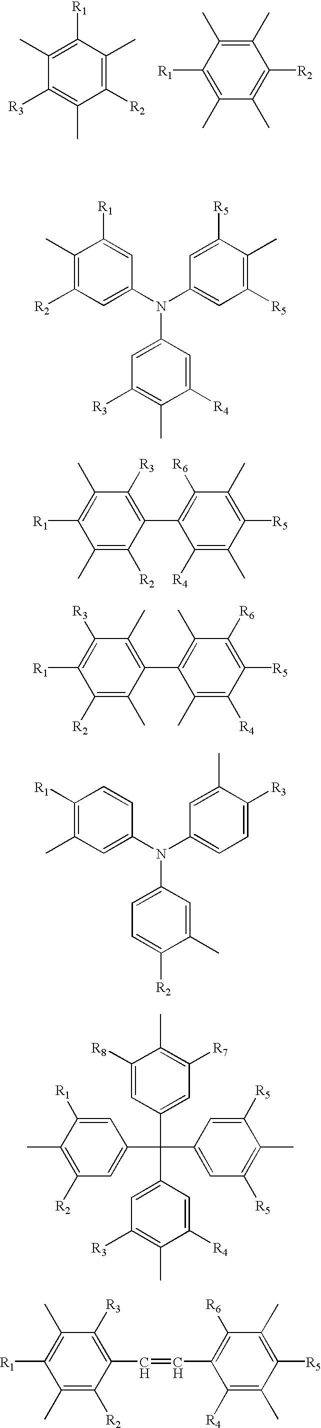
wherein
R₁ to R₁₂ are independently selected from H, an aliphatic group, an aryl group, a halogen, CN and NO₂, and A is O, S, or NR (where R is selected from the moieties defined above for R₁ to R₁₂), and (b) a core moiety having any one of the above ring structures where the bonds for linking to the benzofuranyl groups are at any other positions on the respective ring(s), provided that there are at least three such bonds in all, with the R₁ to Rₓ substituents being correspondingly positioned on the respective ring(s). 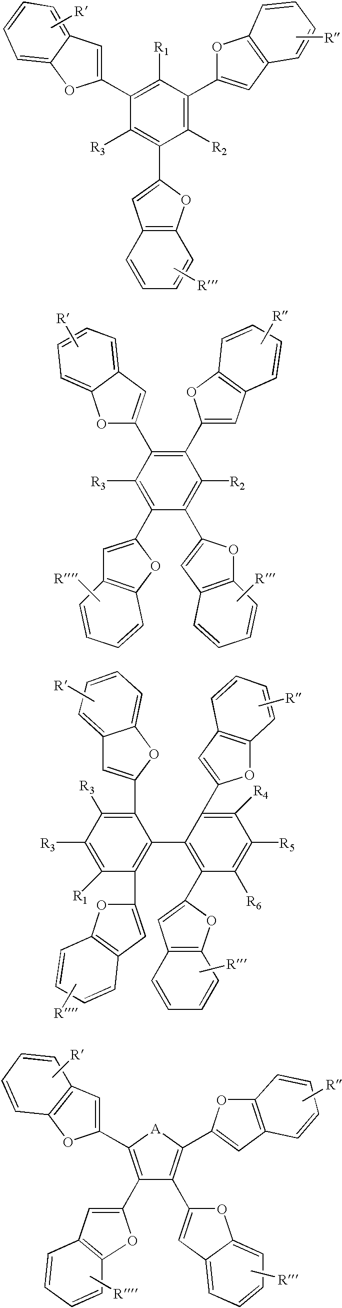
where
each of R₁ to R₈ is independently selected from H, an aliphatic group, an aromatic group, a halogen, CN and NO₂, and each of R′ to R″″ is independently selected from at least one of H, an aliphatic group, an aromatic group, a halogen, CN and NO₂, and A is O, S or NR (where R is selected from the moieties defined above for R₁ to R₈)

5. A method as claimed in claim 1, wherein the branched benzofuran compound prepared is selected from:—

6. A compound having one of the following general formulae:— 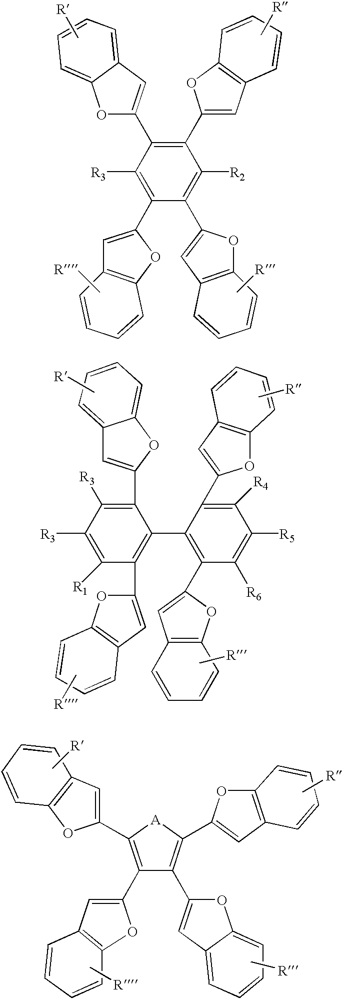
where
each of R₁ to R₈ is independently selected from H, an aliphatic group, an aromatic group, a halogen, CN and NO₂, and each of R′ to R″″ is independently selected from at least one of H, an aliphatic group, an aromatic group, a halogen, CN and NO₂, and A is O, S or NR (where R is selected from the moieties defined above for R₁ to R₈)

7. A compound as claimed in claim 6 having one of the following general formulae: 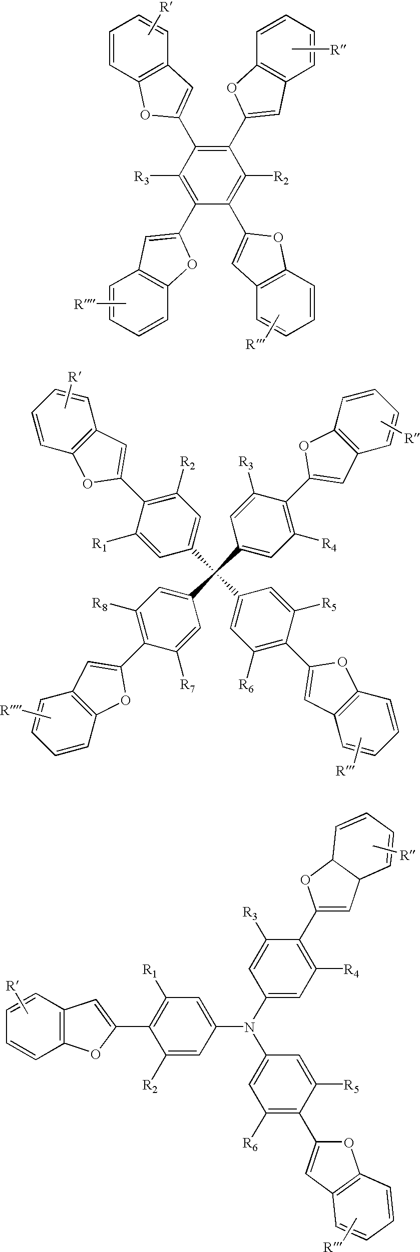
where
each of R₁ to R₈ is independently selected from H, en aliphatic group, an aromatic group, a halogen, CN and NO₂, and each of R′ to R″″ is independantly selected from at least one of H, an aliphatic group, an aromatic group, a halogen, CN and NO₂ and A is O, S or NR (where R is selected from the moieties defined above for R₁ to R₈)

8. The use of a compound when produced by the method as claimed in claim 1, as a charge transport material in an electroluminescent device (for example a laser), in a transistor or in a photovoltaic device.

9. The use of a compound as defined in claim 6, as a charge transport material in an electroluminescent device (for example a laser), in a transistor or in a photovoltaic device.

10. The use of a compound when produced by the method as claimed in claim 1, as a light emitter in an electroluminescent device (for example a laser) in or a photovoltaic device.

11. The use of a compound as defined in claim 6, as a light emitter in an electroluminescent device (for example a laser) in or a photovoltaic device.
